(19) United States
(12) Patent Application Publication
(10) Pub. No.: US 2013/0144144 A1
(43) Pub. Date: Jun. 6, 2013

(54) DEVICE SYSTEM AND METHOD FOR MONITORING AND CONTROLLING BLOOD ANALYTE LEVELS

(71) Applicant: C.G.M.3 LTD., Yokneam (IL)

(72) Inventors: Morris LASTER, Jerusalem (IL); Moshe PHILLIP, Givataim (IL)

(73) Assignee: C.G.M.3 LTD., Yokneam (IL)

(21) Appl. No.: 13/673,198

(22) Filed: Nov. 9, 2012

Related U.S. Application Data
(63) Continuation-in-part of application No. 12/450,919, filed on Feb. 2, 2010, now abandoned.
(60) Provisional application No. 60/996,676, filed on Nov. 29, 2007.
(60) Provisional application No. 60/907,845, filed on Apr. 19, 2007.

(51) Int. Cl. A61M 37/00; A61B 5/145

(52) U.S. Cl. CPC A61M 37/00; A61B 5/14532
USPC 600/365; 600/309; 604/66; 604/504

(57) ABSTRACT

Systems, devices, and methods for monitoring an analyte in a subject. The systems, devices, and methods may include a sensor element being designed and configured for detecting said analyte in blood flowing through a bone of the subject, and a fixation element that is capable of fixating said sensor element within the bone tissue

### CROSS-REFERENCE TO RELATED APPLICATION

This application is a continuation-in-part of U.S. application Ser. No. 12/450,919, filed Feb. 2, 2010, which claims priority to international application PCT/IL2008/000488, filed in English on Apr. 9, 2008, which designated the United States and claims the benefit of priority to U.S. Provisional Patent Application Nos. 60/996,676 filed Nov. 29, 2007 and 60/907,845 filed Apr. 19, 2007, the entirety of each of which is incorporated by reference herein.

### FIELD AND BACKGROUND OF THE INVENTION

The present invention relates to an analyte monitoring device having a bone implanted analyte sensor and, more particularly, to a continuous glucose monitoring system having a bone implanted glucose sensor and infusion pump.

Although diabetes is a chronic condition, it can usually be managed by diet, medications and proper glucose control. The main goal of treatment is to keep blood glucose levels in the normal range. Monitoring blood glucose levels is the best way of managing diabetes. A healthcare provider will periodically order laboratory blood tests to determine the average blood glucose levels via tests such as hemoglobin A1C measurements. While the results of these tests gives an overall sense of how blood glucose levels are controlled daily functional control of blood glucose levels and treatment requires that patients monitor their own blood glucose levels frequently between six and ten times a day

Numerous devices for home monitoring of glucose levels are known in the art. The most popular devices currently in use employ a lancet for pricking skin to draw a drop of blood and test strips which are read by an optical reader. Although such devices are accurate, they necessitate periodic skin pricking which may produce discomfort to the tested individual. In addition, such devices cannot provide continuous blood glucose monitoring which is important to diabetic individuals and are necessary for real time medicinal and dietetic adjustments to glucose levels

To overcome these problems, non-invasive monitoring devices or implantable continuous monitoring devices have been proposed.

Non-invasive glucose sensing is the ultimate goal of glucose monitoring, but the most investigated non-invasive approach utilizing near-infrared (NIR) spectroscopy, is presently too imprecise for clinical application (there is not even one single noninvasive techniques in clinical use). Thus, non-invasive glucose monitors (e.g. GlucoWatch G2 Biographer, manufactured by Cygnus Inc.) require daily invasive measurements in order to maintain calibration. In addition, since such devices tend to be less accurate than invasive glucose measurements, doctors recommend that periodic conventional blood glucose monitoring be used along with such devices.

To traverse the limitations of NIR glucose monitoring, interstitial fluid monitoring devices have been developed.

Percutaneous monitoring devices utilize iontophoresis to sample the interstitial fluid without breaking the skin surface. The accuracy of such devices is influenced by skin temperature and perspiration and as such use thereof for continuous glucose monitoring is limited.

Implanted monitoring devices typically employ a sensor which is implanted subcutaneously. Implantable glucose sensors typically utilize an amperometric enzyme probe or an optical probe which measure the level of glucose in the interstitial fluid surrounding the tissue every several seconds and relay the information via wires (e.g. Minimed™, Medtronics) or wirelessly (SMSI™ Glucose Sensor, Sensors for Medicine and Science) to a monitor which is carried by the user.

Continuous glucose monitoring devices provide information about the direction, magnitude, duration, frequency, and causes of fluctuations in blood glucose levels. Compared with non-implanted glucose monitors, continuous monitoring devices can provide more detail with respect to glucose trends and thus help identify and prevent unwanted periods of hypo- and hyperglycemia.

Although implanted monitors are more accurate than non-invasive monitors they suffer from several limitations. Since the body tries to isolate any implanted objects by tissue remodeling, glucose transport to the sensor can be reduced. In addition, the glucose levels in the interstitial fluid do not always accurately reflect blood glucose levels since several physiological factors might influence the interstitial glucose levels (Steil et al. Diabetes Techn and therape (5):1, 2003 and Schmidtke et al. Proc. Natl. Acad Sci USA 95:294-9, 1998) and since glucose levels in the interstitial fluid can lag or lead blood glucose levels by several minutes. Such factors can severely limit the accuracy of implanted sensors and thus limit their use especially in cases where glucose monitoring is utilized for closing the loop on insulin delivery in systems for controlling glucose levels. Additionally, these devices involve the use of expensive cartridges which need to be replaced daily or every few days.

A further problem associated with all continuous analyte measurement systems that utilize indwelling detectors is that the useful life of such systems is often limited due to the instability of the sensor at its site of implantation within the host, for example, by damage to the detector that is caused both by direct contact with the rapidly flowing blood stream (in the case of intravascular devices) and, more generally, by the response of the body to the presence of foreign body. Such responses include non-specific inflammatory states and the associated production of granulation tissue and fibrosis, as well as more specific immune reactions. As mentioned above, these responses can be so severe as to restrict the usefulness of implanted, indwelling electrodes and other implants.

In view of the manifold advantages of the continuous glucose monitoring system disclosed in the aforementioned co-owned international patent application, as well as the advantages of many other systems that use indwelling analytical sensors, there is a clear and pressing need for a technical solution to this problem of the poor stability of the implanted sensor in the face of the various mechanisms used by the body to deal with the ‘threat’ posed by the presence of this foreign body.

A further technical requirement of indwelling analyte measurement systems of this type is the need for the sensor to remain in close contact with vascular tissue (i.e. in an oxygen-rich environment), despite the attempts of the patient's body to surround said sensor with fibrotic tissue. In view of this additional requirement, it is not possible to solve the abovementioned problem of the sensor being attacked by host defense mechanisms by means which would lead to isolation of the sensor from vascular tissue. On the contrary, an ideal solution would actually modulate the location of blood and vascular tissue into the region occupied by the sensor.

An additional requirement of long-term indwelling analyte measurement devices is that there needs to be adequate provision for preventing any movement of the detector following its implantation at the desired site, in order to obviate problems such as sensor movement or removal, and to prevent shear force movement or damage to sensor, sensor malfunction and/or bleeding at the implantation site.

A need thus exists for a device, system and method for monitoring and controlling glucose levels that is devoid of all of the above limitations.

### SUMMARY OF THE INVENTION

According to one aspect of the present invention there is provided a device for monitoring an analyte in a subject comprising a sensor element being designed and configured for detecting the analyte in blood flowing through bone of the subject.

According to further features in preferred embodiments of the invention described below, the sensor element is designed and configured for implantation within bone tissue.

According to still further features in the described preferred embodiments the sensor element is designed and configured for implantation within cancellous tissue of the bone.

According to still further features in the described preferred embodiments the sensor element is designed and configured for implantation within periosteum tissue of the bone.

According to still further features in the described preferred embodiments the sensor element is designed and configured for implantation within compact bone tissue of the bone.

According to still further features in the described preferred embodiments the sensor element is designed and configured for implantation within Haversian canals (osteons).

According to still further features in the described preferred embodiments the device further comprises a power source for powering the sensor element.

According to still further features in the described preferred embodiments the device further comprises circuitry for remotely powering the sensor element.

According to still further features in the described preferred embodiments the analyte is selected from the group consisting of urea, ammonia, hydrogen ions, minerals, enzymes, and drugs.

According to still further features in the described preferred embodiments the analyte is glucose.

According to still further features in the described preferred embodiments the sensor element is an electrochemical or an optical sensor element.

According to still further features in the described preferred embodiments the sensor element includes a membrane selective for the analyte.

According to still further features in the described preferred embodiments the cage housing the sensor element includes non-osteoconductive material.

According to another aspect of the present invention there is provided a system for monitoring an analyte in a subject comprising a device including a sensor element being designed and configured for detecting the analyte in blood flowing through a bone of the subject and a control unit for controlling the device.

According to still further features in the described preferred embodiments the sensor element is designed and configured for implantation within bone tissue.

According to still further features in the described preferred embodiments the sensor element is designed and configured for implantation within cancellous tissue of the bone.

According to still further features in the described preferred embodiments the sensor element is designed and configured for implantation within periosteum tissue of the bone.

According to still further features in the described preferred embodiments the sensor element is designed and configured for implantation within compact bone tissue of the bone.

According to still further features in the described preferred embodiments the sensor element is designed and configured for implantation within Haversian canals.

According to another aspect, the present invention provides means for preventing the sensor element from being attacked by host defense mechanism, while permitting said element to remain in contact with vascular tissue. Said means are provided in the form of a rigid, cage-like implant-protecting device having a body perforated by a longitudinally-disposed central bore of a size and form that permits the insertion and retention of a sensor element such as an analyte detector or electrode. Generally (but not always), said central bore is full-length, passing from one end of the device to the other, penetrating the distal and proximal extremities thereof.

According to yet another aspect, the present invention provides one or more fixation elements for physically stabilizing and fixating the sensor element within the bone marrow. In one preferred embodiment, said fixation element comprises a band constructed from a biocompatible material, said band being capable of being attached to both cortical bone (by means of itself having being a screw type element, fixation by plate/screws or other retaining elements), and to the upper portion of the sensor element. In another preferred embodiment, the fixation element comprises one or more biocompatible screws that are capable of being attached to cortical bone and to the sensor element, thereby directly stabilizing and fixating the sensor element within the bone marrow.

According to still further features in the described preferred embodiments the device and the control unit are designed for wireless communication.

According to still further features in the described preferred embodiments the wireless communication is mediated via magnetic, electromagnetic or acoustic energy.

According to still further features in the described preferred embodiments the device is wired to the control unit.

According to still further features in the described preferred embodiments the device includes a power supply.

According to still further features in the described preferred embodiments the device includes an induction coil.

According to still further features in the described preferred embodiments the analyte is selected from the group consisting of urea, ammonia, hydrogen ions, minerals, enzymes, and drugs.

According to still further features in the described preferred embodiments the analyte is glucose.

According to still further features in the described preferred embodiments the sensor element is an electrochemical or an optical sensor element.

According to still further features in the described preferred embodiments—the sensor element includes a membrane selective for the analyte.

According to still further features in the described preferred embodiments the sensor element includes non-osteoconductive material.

According to yet another aspect of the present invention there is provided a method of monitoring an analyte in a subject comprising detecting the analyte in blood flowing through bone tissue of the subject thereby monitoring the analyte in the subject.

According to still further features in the described preferred embodiments detecting is effected by implanting an analyte sensor in a bone of the subject.

According to yet another aspect of the present invention there is provided a system for controlling blood glucose levels in a subject comprising: (a) a sensor element being designed and configured for detecting the analyte in blood flowing through a bone of the subject; and (b) a reservoir for providing to the blood flowing through the bone of the subject at least one composition capable of modifying a level of glucose.

According to still further features in the described preferred embodiments the sensor element is designed and configured for implantation within bone tissue.

According to still further features in the described preferred embodiments the reservoir is in fluid communication with a port/catheter attached to tissue of the bone.

According to still further features in the described preferred embodiments the system further comprises a mechanism for pumping the composition from the reservoir to the blood flowing through the bone.

According to still further features in the described preferred embodiments the system further comprises a power source for powering the sensor element and the mechanism.

According to still further features in the described preferred embodiments the mechanism utilizes peristalsis, a propellant, osmotic pressure, a piezoelectric element or an oscillating piston/rotating turbine.

According to still further features in the described preferred embodiments the sensor element is an electrochemical or an optical sensor element.

According to still further features in the described preferred embodiments the reservoir further includes a filling port.

According to still further features in the described preferred embodiments the reservoir is intracorporeal or extracorporeal.

According to still further features in the described preferred embodiments the at least one composition is insulin and/or glucagon.

The present invention successfully addresses the shortcomings of the presently known configurations by providing a system which enables real-time accurate monitoring and controlling of glucose levels.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is a drawing illustrating bone anatomy.

FIG. 1b illustrates the iliac crest bone.

FIGS. 2a-b illustrate a system for continuous glucose monitoring constructed in accordance with the teachings of the present invention and implanted in an axial skeleton bone.

FIGS. 3a-b illustrate several embodiments of a system of controlling the level of glucose in the blood of a subject.

FIGS. 4a-c are graphs illustrating glucose levels in blood drawn from a vein or bone marrow of rabbits following administration of dextrose or insulin; Red line—vein blood; Blue line—bone derived blood.

FIGS. 5 to 10 depict several different preferred embodiments of the device of the present invention.

FIGS. 11 to 15 are histological sections of an in situ cage-like device of the present invention, eight weeks following implantation into the sternum of a pig.

FIGS. 16 to 18, 19, 21 and 23 depict some further preferred embodiments of the cage-like device of the present invention.

FIGS. 20, 22 and 24 are histological sections of the cage-like devices shown in FIGS. 19, 21 and 23 (respectively), eight weeks following implantation into the sternum of a pig.

FIG. 25 provides a cross-sectional view of an analyte sensor of the present invention following implantation within bone marrow and fixation by means of a titanium band that is secured within cortical bone.

FIG. 26 illustrates an alternative embodiment of the sensor fixation means, comprising an in-line titanium screw that is inserted into the cortical bone.

FIG. 27 depicts yet another embodiment of the sensor fixation means wherein said means comprise two biocompatible screws that pass through the upper part of the sensor and then into cortical bone.

### DESCRIPTION OF THE PREFERRED EMBODIMENTS

The present invention is of an analyte monitoring device and system which can be used to continuously monitor blood analyte levels and thus provide a monitored subject with data relating to real-time analyte levels, trends in analyte levels and the like.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description and example or illustrated in the drawings. The invention is capable of other embodiments or of being practiced or carried out in various ways. Also, it is to be understood that the phraseology and terminology employed herein is for the purpose of description and should not be regarded as limiting.

Monitoring of glucose levels is the main goal of continuous analyte monitoring technologies. Although numerous attempts have been made to produce a reliable continuous glucose monitoring device, the reality is that at present day no implanted continuous monitoring device is commercially marketed as stand-alone solution.

Prior art implanted glucose monitors suffer from several limitations which result from the site of implantation. Subcutaneous implantation of glucose monitors can lead to implant encapsulation while accuracy of such devices is limited by the fact that ISF glucose levels sampled by such devices do not mirror those of blood. On the other hand, while blood vessel coupled glucose monitors are more accurate, attachment thereof to blood vessels such as veins can lead to systemic infections, blood flow perturbations, clotting, generation of emboli, and tissue reactions to the implant.

While reducing the present invention to practice, the present inventors have devised an analyte sensor which directly monitors blood analyte levels and yet does not suffer from the limitations of blood vessel-coupled analyte sensors.

As is further detailed herein, the present device is designed and configured for detecting analytes within blood flowing through a bone tissue. Blood flow through bone marrow has been shown to be an accurate real time mirror of systemic blood measurements [Hurren J S, Burns. 2000 December; 26 (8):727-30; Ummenhofer et al Resuscitation. 1994 March; 27 (2):123-8) and Example 2 hereinbelow]. Bone-attachment of an analyte sensor minimizes the possibility of infection, migration or movement of the analyte sensor, tissue reaction to the implant (encapsulation) and generation of emboli while enabling sampling of blood fluids with minimal flow perturbations.

Thus, according to one aspect of the present invention there is provided a device for monitoring an analyte in a subject.

The device of the present invention includes a sensor element(s) which is designed and configured for detecting the analyte in blood flowing through a bone of the subject.

The term “analyte,” as used herein, refers to a substance or chemical constituent which is present in a biological fluid (e.g. blood) and can be monitored (e.g. quantified and/or qualified). Analytes can include naturally occurring substances, artificial substances, to metabolites, and/or reaction products. Preferably, the analyte for monitoring by the device of the present invention is glucose. However, other analytes are contemplated as well, including but not limited to, PH, electrolytes, CO₂ and O², ammonia, acetone and beta-hydroxy-butyrate, acetoacetate, lactate, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), acarboxyprothrombin; acylcamitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carbon dioxide; carnitine; camosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobinopathies, A,S,C,E, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17 alpha-hydroxyprogesterone; hypoxanthine phosphoribosyl transferase; immunoreactive trypsin; lactate; lead; lipoproteins ((a), B/A-1, β); lysozyme; mefloquine; netilmicin; oxygen; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, pH, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins and hormones naturally occurring in blood or interstitial fluids may also constitute analytes in certain embodiments. The analyte may be naturally present in the biological fluid, for example, a metabolic product, a hormone, an antigen, an antibody, and the like. Alternatively, the analyte may be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine.

The device of the present invention can be implanted within any bone of the subject. Preferred bones are pelvis and sternum, vertebral bodies and long bones.

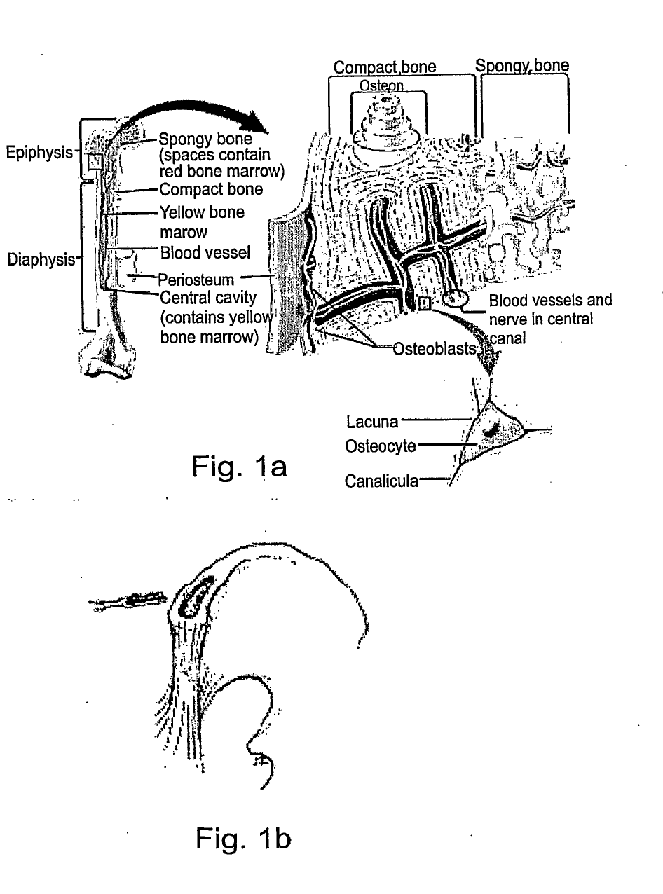

FIG. 1a schematically illustrates anatomy of a bone showing the various bone tissue regions. FIG. 1b illustrates an iliac crest with cortex removed, exposing bone marrow comprised of cancellous bone. Bone marrow is a naturally occurring arterio-venus shunt and thus is highly suitable for placement of an analyte sensor, in particular a continuous, real time glucose sensor.

The present device can be partially or fully implanted within any tissue region of a bone including cancellous tissue, periosteum tissue and compact bone tissue.

Implantation can be effected via any one of numerous approaches used to access bone tissue, including for example, various drilling or cutting approaches. Such approaches are well known to the ordinarily skilled artisan and as such no further description of such approaches is provided herein.

The present device is designed such that when it is implanted to bone tissue, the sensor element(s) resides within the intra-medullary/intra-bone marrow blood sinus present within bone tissue. This enables the sensor element(s) to sample blood flowing through the bone tissue and to provide accurate and real-time analyte monitoring.

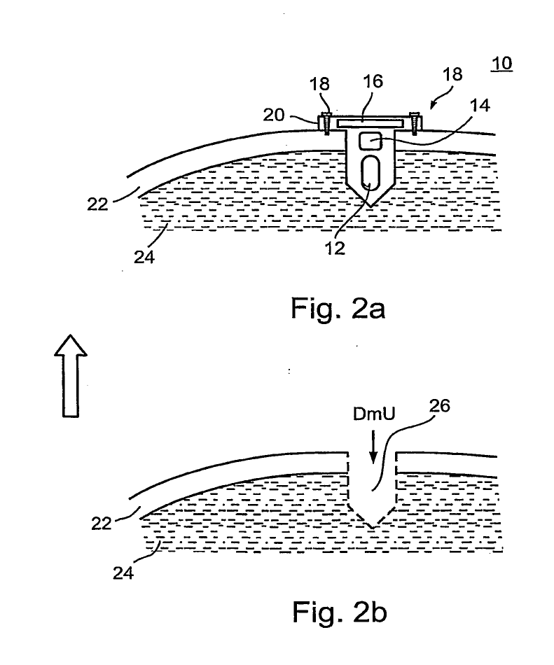

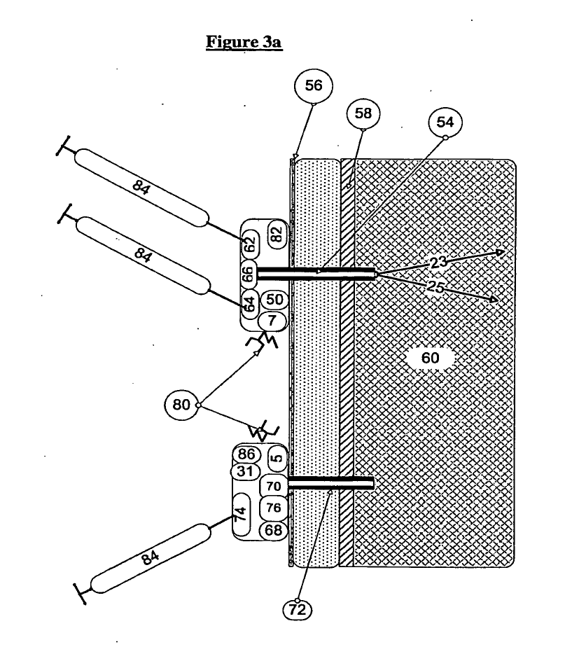

The present device can be of any shape and size suitable for bone attachment. The shape and size of the present device will largely depend on whether the device is partially or fully implanted within the bone, the site of implantation and the type of communication between the device and a controller unit (further described hereinbelow). In general, the device can be spherical, cylindrical, rectangular or in a shape having a diameter/width of 1 mm-2.5 cm and a length of 5 mm-5 cm. FIG. 2a which is described in greater detail Examples section which follows illustrates one preferred device configuration.

In a configuration in which the device is partially implanted within bone, the sensor element(s) component of the device is configured such that it extends into the bone tissue and contacts the blood flowing within intra-medullary/intra-bone marrow blood sinus, while the device body which houses additional components such as power source, circuitry, communications devices (e.g. coils, antennas) and the like can be placed within soft tissues surrounding the bone or it can be attached to the bone surface via attachment anchors suitable for bone anchoring. Bone anchor configurations suitable for use with the present device include bone screws/plates and the like. Soft tissue anchoring can be effected via sutures staples or anchors using approaches well known in the art.

In the partially implanted configuration of the present device, the sensor element(s) can be fitted into a small hole/slit which is drilled or cut into the bone. Such a hole or slit is long enough to extend through the cortex and into cancellous bone. For example, in a device configured for use in long bones, a hole 5 mm-5 cm mm long and 1 mm-2.5 cm in diameter can be drilled into the bone and used to accommodate the sensor element(s) of the present device.

Since a partially implanted configuration requires minimal bone drilling/cutting, such a configuration is highly suitable for smaller bones which cannot accommodate the entire device. Examples of such bones include vertebral bodies, sternum, and the like.

A fully implanted configuration in which the entire device is implanted within the bone is also contemplated herein. In such a configuration, the device body is implanted into the bone tissue and the sensor element(s) is exposed to the blood flowing therein. As is well known in the art, implantation of foreign objects (e.g. orthopedic implants) within bone is well tolerated by the body and produces minimal body reactions as compared to implantation within soft tissues. Thus, a fully implanted configuration is advantageous in that the device body is fully encapsulated by bone tissue and less exposed to possible tissue reactions that could lead to encapsulation, biofilm formation erosion and the like.

In the fully-implantable embodiments of the device of the present invention, it is, in most cases, necessary to provide adequate fixation and mechanical stabilization of the sensor element, in order to prevent movement, displacement, malfunction and/or unwanted removal of the sensor. In addition, poor fixation of the sensor element could—as a result of its undesired mobility at the implantation site—lead to trauma and bleeding at the implantation site. Thus, in some embodiments, the presently-disclosed device further comprises a fixation element for fixating the sensor element within the bone tissue.

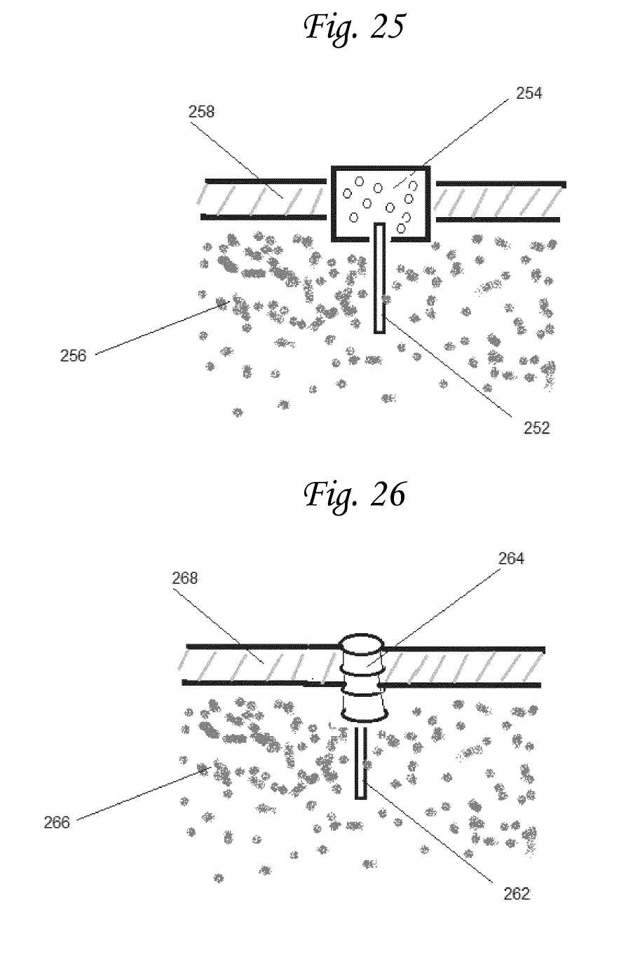

In one such preferred embodiment, said fixation element comprises a porous band constructed from a biocompatible, bone-growth promoting material, such as porous titanium. An example of this type of fixation element is shown in FIG. 25, in which the porous band 254 is seen to be attached to cortical bone 258 overlying the implantation site in the bone marrow 256 (by means of screws or other retaining elements), and to the upper portion of the sensor element 252. The efficiency of the porous band as a fixation device increases with time, as a result of ingrowth of osseous tissue into the pores.

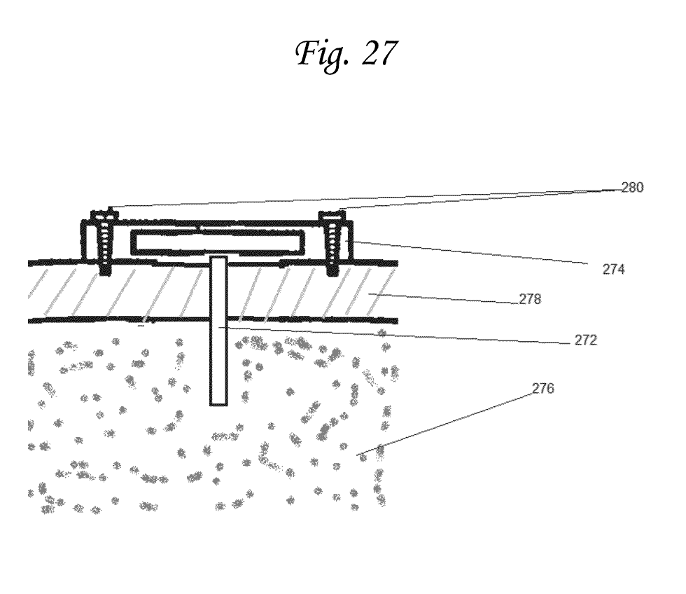

In other preferred embodiments, the fixation element comprises one or more biocompatible screws that are capable of directly stabilizing and fixating the sensor element within the bone marrow. Thus, FIG. 26 demonstrates the use of a single, centrally-placed screw (preferably manufactured from titanium) 264 inserted through cortical bone 268 into bone marrow 266. Sensor element 262 is attached to the lower portion of screw 264, and is stabilized and fixated thereby. FIG. 27 illustrates an alternative embodiment of this type in which sensor element 272, embedded within one marrow 276 is attached to thin fixation plate 274 (constructed from a biocompatible material such as titanium) which is itself firmly attached to cortical bone 278 by means of small fixation screws 280.

As mentioned hereinabove, one potential problem associated with continuous analyte measurement systems that utilize indwelling detectors is that the useful life of such systems is often limited due to the instability of the sensor at its site of implantation within the host, for example, by damage to the detector that is caused both by direct contact with the rapidly flowing blood stream (in the case of intravascular devices) and, more generally, by the response of the body to the presence of foreign body. Such responses include non-specific inflammatory states and the associated production of granulation tissue and fibrosis, as well as more specific immune reactions. These responses can be so severe as to restrict the usefulness of implanted, indwelling electrodes and other implants.

A further technical requirement of indwelling analyte measurement systems such as the system provided by the present invention is the need for the sensor to remain in close contact with vascular tissue (i.e. in an oxygen-rich environment), despite the attempts of the patient's body to surround said sensor with fibrotic tissue. In view of this additional requirement, it is not possible to solve the abovementioned problem of the sensor being attacked by host defense mechanisms by means which would lead to isolation of the sensor from vascular tissue. On the contrary, an ideal solution would actually encourage the ingrowth of vascular tissue into the region occupied by the sensor.

This aspect of the present invention provides a practical solution to the aforementioned technical problems. This solution is provided in the form of a rigid, cage-like implant-protecting device having a body perforated by a longitudinally-disposed central bore of a size and form that permits the insertion and retention of a sensor element such as an analyte detector or electrode. Generally (but not always), said central bore is full-length, passing from one end of the device to the other, penetrating the distal and proximal extremities thereof.

While experimenting with several implant designs the present inventors identified design parameters which enable fabrication of an implant that enables long term use of a sensor carried thereby without the aforementioned limitations.

Such parameters include, but are not limited to:

(i) an implant lumen that creates a reservoir capable of fluid communication with surrounding vascular tissue and circulation of blood therethrough;

(ii) implant lumen openings that can be covered by growth of bone marrow tissue which serves as a source of blood (and vascular tissue)

(iii) a central lumen which extends from an opening in the proximal end to an opening in the distal end of the implant facilitating sensor positioning and replacement following bone implantation of the implant device;

(iv) a central lumen which includes fluid communication channels preferably angled with respect to a longitudinal axis of the implant thereby increasing circulation through the reservoir;

(v) an external surface which facilitates bone anchoring while minimizing radial outward forces on the bone tissue

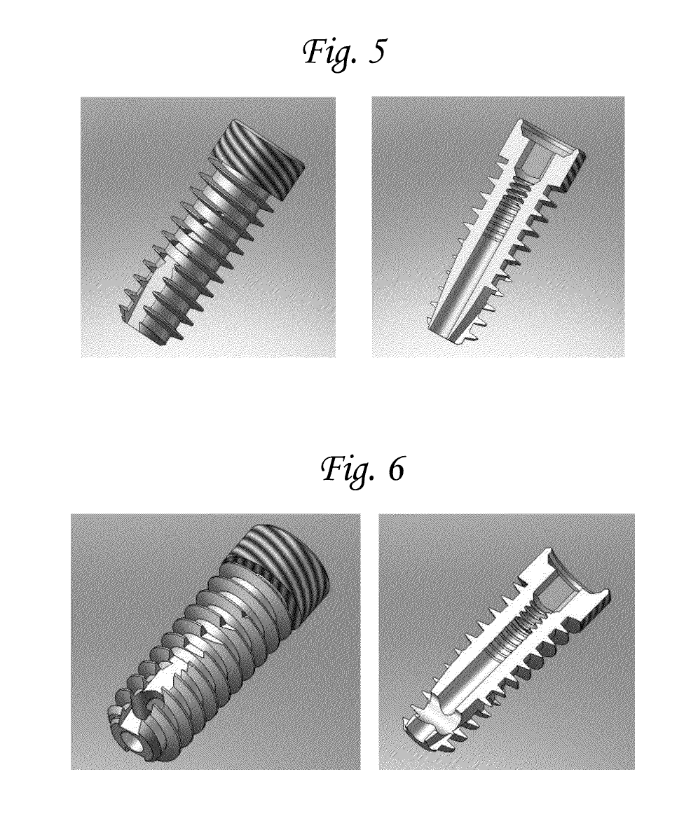

Several exemplary configurations of the device of the present invention designed in accordance with the parameters described above, as well as the implantation thereof into experimental animals will be described in more detail hereinbelow, with reference to the accompanying drawings (principally, FIGS. 5-24).

Preferably, the device mentioned hereinabove is elongate in form, having a longitudinal axis which is significantly longer than its width or diameter. The cross-sectional shape of the elongate body of the device is typically round, but it may also be any other suitable shape including (but not limited to) elliptical, square, rectangular, triangular and so on. The elongate body typically has either a cylindrical or slightly-tapering, conical form when viewed from the side. Generally, the elongate device bears a screw thread on its external surface. In one preferred form of the device, the external screw thread extends from the distal extremity of the device proximally, ending a short distance from the proximal extremity thereof. The non-threaded proximal portion thereby constitutes as a proximal head region.

In a particularly preferable form, the cage-like device is provided in the form of an elongate screw having at least one central bore in the form of an internal passage orientated parallel to the longitudinal axis of said screw, wherein said passage (also referred to herein as the central bore) passes along the entire length of the device and penetrates both the proximal and distal tips thereof. In some particularly preferred embodiments, the device also possesses at least one lateral channel that is orientated such that it runs in a direction that is not parallel to the longitudinal axis of the device, and preferably at right angles thereto.

These channels may either be partial-depth channels (i.e. piercing the external wall of the device at one side and terminating in the longitudinal passage) or through-and-through channels that pierce the external wall on one side of the device, perforates the internal wall defining the internal longitudinal passage (central bore) and then continue to pierce the external wall on the other side.

While in some embodiments of the device, there may be only one lateral channel, in other versions, two or more such channels may be present. In one preferred embodiment, the device has two lateral channels. In the case that the device has two or more lateral channels, it has been found that it is preferable for the distance between adjacent lateral channels (i.e. measured along the longitudinal axis) to be no greater than 5 mm, and even more preferable for this distance to be no greater than 2 mm.

Typically, the internal diameter of the lateral channels is in the order of 0.15 to 5.0 mm.

In another embodiment, the device may comprise a plurality of lateral channels, preferably arranged in the form of an evenly-spaced array. In such a case the internal diameter of each of said channels in the array will be substantially less than the diameters of the aforementioned channels that are present either singly or in pairs or triplets, for example, in the range of 150 to 900 μm.

The primary function of the lateral channels is to permit blood, serum and blood-related tissue such as bone marrow to enter the internal spaces of the cage device of the present invention, thereby enabling a sensing device (such as a glucose electrode) placed within the longitudinal passage to be in contact with said blood and related tissues. In this manner, the sensing device may perform measurements of analyte concentrations in a way which is representative of the blood concentrations found throughout the circulatory system. The present inventors have also found that the internal diameter of the lateral channels may have a significant impact on the tissue that is capable of entering said channels thereby being accessible to the sensing device. Thus, as a general rule, small-diameter lateral channels, such as those having an internal diameter of 0.2 mm or less, do not permit ingress of bone marrow tissue. Rather, the internal spaces of the cage device tend to fill with blood and serum. On the other hand, lateral channels having internal diameters greater than about 0.5 mm permit the ingress of bone marrow tissue (as well as the liquid blood components). In certain circumstances, this may be highly desirable, since the establishment of an organized bone marrow structure within the internal space of the cage-device (and in fluid contact with the blood vessels and bone marrow located outside of said device), may permit the establishment of favorable conditions for providing blood to the sensor device, such that the latter is able to perform representative analyte assays over a long period of time.

In some embodiments, as will be described in more detail hereinbelow, the cage-like device further comprises one or more additional longitudinal channels entirely enclosed within the wall thereof. In this alternative embodiment, the additional longitudinal channel(s) may be used to house the sensor device (such as an analyte detector or electrode).

It has also been found advantageous, in some circumstances, for the internal walls of the device that define the longitudinal internal passage and/or the lateral channels to be smooth-bored, for example polished. As will be described further hereinbelow, highly polished interior surfaces may prevent the attachment and organization of bone marrow tissue within the device, even when the lateral apertures are of a sufficiently large diameter to permit ingress of such tissue.

In other circumstances, the reverse may be true, that is the internal wall of the longitudinal passage may advantageously be roughened, for example by means of cutting grooves therein. This roughening process has the desirable effects of improving the stability of the implant, permitting full tissue-implant integration and stability. In addition, it assists in minimizing fibrous tissue formation and maintaining an oxygen-enriched environment. Such rough walls may be used, for example, when it is desired to encourage the persistence and organization of bone marrow tissue within the interior of the implanted device.

Furthermore, it has also been found advantageous to coat the internal wall of the longitudinal passage with an anti-fibrosis coating. One example of such a coating is the heparin-containing composition known commercially as Excor (Carmeda, Canada).

The implant devices of the present invention may be constructed of one or more of the biocompatible materials selected from the group consisting of titanium, stainless steel, Nitinol or biocompatible polymers and plastics such as nylon, PEEK and polyethylene.

In a particularly preferred embodiment, the device is constructed of titanium.

The implant devices may be constructed using any of the suitable manufacturing techniques well known in the art including (but not limited to) machining, die-casting, laser cutting and etching. Polishing of the internal cavities and channels of the devices may be achieved using electro-polishing, reaming and/or brushes, with or without the use of smoothing pastes.

The length of the implant devices of the present invention is generally in the range of 10 to 25 mm, while the external diameter thereof is generally in the range of 1.5 to 10 mm. The longitudinal internal channel preferably has an internal diameter in the range of 1 to 6 mm.

In one particularly preferred embodiment, the device length is 16 mm, the external diameter is 6 mm and the diameter of the central bore is 5 mm.

The present invention is further directed to methods for implanting a sensor element in a protected environment within bone tissues of a subject. The term ‘protected environment’ refers to the manner in which the sensor element is allowed to come into contact with liquid blood and/or bone marrow tissue (in order that said sensor element may be used to make analyte concentration measurements that are representative of the concentration of said analytes within the blood stream of the subject), while at the same time inhibiting or preventing (either fully or partially) contact of fibrous tissue with said sensor element. The cage-like devices may be used to protect implanted electrodes within bony tissue using either of the two following general approaches:

1) Surgical access to the desired implant site (e.g. iliac crest, sternum) is achieved, and a hole, of a size similar to the external diameter and length of the selected cage-like device, is drilled therein. An implant cage of the present invention having the upper (proximal) end of the central bore opening closed with a plug is then inserted into said drilled hole and retained therein by friction, by the use of laterally-placed retaining screws or by means of gluing using biocompatible adhesives. At a certain time following implantation of the cage, the central bore plug is removed and an analyte sensor device is inserted into the central bore.

2) Surgical access and drilling of the placement hole is performed as described above. The upper plug is removed from the device and an analyte sensor device is inserted into the central bore (or alternatively, in some embodiments, into one or more additional longitudinal channels formed within the device wall). The cage-like device containing the pre-inserted sensor is then fitted into the pre-drilled hole and retained in place as indicated above.

In one preferred embodiment, the sensor element used in the methods of the present invention is a glucose-detecting sensor.

Examples of suitable analyte-measuring electrodes that may be used in conjunction with the implant-protecting cage-like device and method of the present invention include: the DexCom SEVEN PLUS sensor, the Medtronic MiniMed sensor and the Abbott FreeStyle Navigator sensor.

In another aspect, the present invention is directed to a system for implanting a sensor element (including but not limited to an analyte detector or an elongate electrode) within a body tissue comprising an implant-protecting device according to any one of the preceding claims and a sensor element, wherein said sensor element is capable of being inserted, either fully or partially, within an internal channel of said implant-protecting device. In a preferred embodiment, the sensor element is a glucose-sensing device.

It is to be noted that the implant-protecting cage devices disclosed herein are not solely intended to be used as cages for enclosing and protecting indwelling sensor devices. Rather, they may be used in any circumstance in which there it is necessary to encourage and support the ingrowth of vascular tissue into an implanted device and to prevent or inhibit the ingress of fibrotic tissue therein. Many types of implanted device that are commonly used in orthopedics, general surgery and dentistry share this dual requirement, and the cage-like devices of the present invention, with the specially-designed structural features described hereinabove may be used in the place of (or in conjunction with) the conventional implants that are in current clinical use. Thus it may be appreciated that the present invention also includes within its scope an implantable device (as disclosed and described hereinabove in all of its aspects and with all of its features) that comprises a tissue anchoring element containing at least one internal lumen, wherein said internal lumen is configured (as described in detail hereinabove) such that it is capable of providing a reservoir suitable for receiving and holding a biological fluid (e.g. blood). The boundaries of said reservoir may be defined either entirely by the walls of the internal lumen, or by at least a portion of the surface of said walls together with the cells of the tissue into which said device has been implanted. As disclosed above (and explained in more detail hereinbelow), the various physical features of the device of the present invention (e.g. the number, dimensions and position of the lateral channels as well as the surface properties of the internal walls defining the longitudinal and lateral channels) may be altered in order to obtain a device which, upon implantation into (for example) bone tissue, functions as a semi-permeable tube thereby permitting the ingress of specific tissues into its internal channels. As a result, these internalized tissues and fluids may be used—together with the material of the implanted device itself—to define an internal space or reservoir. In this regard, the various structural features of the implantable device itself are selected in accordance with some or all of the functional parameters listed above. Of particular advantage are parameters that relate to:

(i) limiting migration into the internal spaces of the device of either all cell and tissue types, e.g. by limiting the size of the lateral channels or by limiting—via surface chemistry—the ingress of specific tissues (e.g. of fibrotic tissue but not of bone-marrow tissue); and

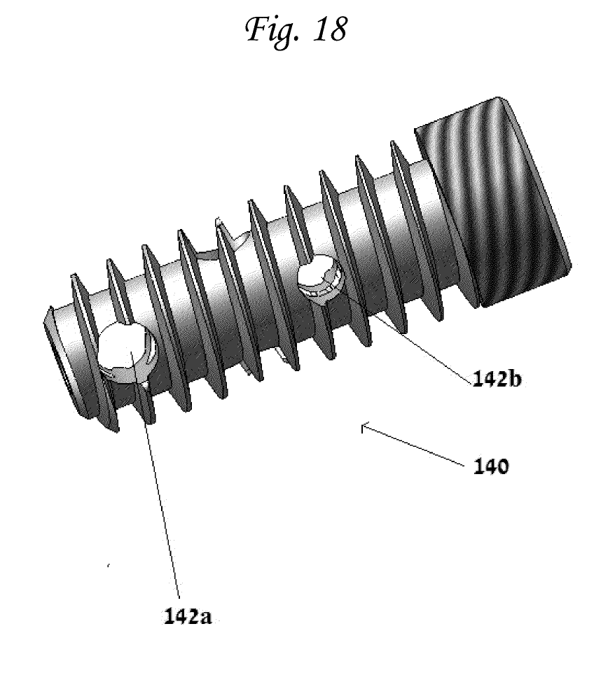

(ii) enabling the flow of the desired biological fluid—such as blood—into said lumen, directing the flow and controlling the flow-rate and fluid pressure therein, thereby providing an optimal analyte-sensing environment. This may be achieved for example by selecting a device having two lateral channels with different internal diameters. As will described hereinbelow (with reference to the embodiment illustrated in FIG. 18) this arrangement leads to the establishment of a circulatory blood flow, whereby blood and serum enter one channel, pass through the central bore and then leave the internal spaces of the device through the other lateral channel. This arrangement may be particularly advantageous when the device of the present invention is used to sample or assay analytes within the blood circulation, for which fluid pooling may be problematic (e.g. for hemodynamic reasons such as settlement and aggregation of certain blood cells and solutes such as certain proteins and other solutes).

Some specific embodiments of the cage device of this aspect of the present invention, as well as the results of studies in which some of said embodiments were implanted into experimental animals, are disclosed and described in Examples 4-6, hereinbelow.

As is mentioned hereinabove, the device of the present invention includes a sensor element(s) which is designed for detecting an analyte of interest.

Such a sensor is preferably chemical or optical in nature. Chemical sensors used for analyte detection are typically amperometric enzymatic sensors.

A typical amperometric enzymatic sensor element(s) includes a non-conductive housing, a working electrode (anode), a reference electrode, and a counter electrode (cathode) passing through and secured within the housing thus forming an electrochemically reactive surface at one location on the housing and an electronic connective means at another location on the housing. The sensor element(s) also includes a membrane affixed to the housing and covering the electrochemically reactive surface. The counter electrode generally has a greater electrochemically reactive surface area than the working electrode. During operation of the sensor, a blood sample or a portion thereof contacts (directly or after passage through the membranes) an enzyme (for example, glucose oxidase in the case of glucose monitoring). The reaction of the analyte and the enzyme results in the formation of reaction products that allow a determination of the analyte (e.g., glucose) level in the blood sample.

The sensor element(s) can be shaped as a cylinder or a thin film; typical thin film electrochemical sensors are described in U.S. Pat. Nos. 5,390,671, 5,391,250, 5,482,473 and 5,586,553.

Three general strategies are used for the electrochemical sensing of an analyte, all of which use an immobilized form of an enzyme that catalyzes the oxidation of the analyte.

For example, in the case of glucose, glucose oxidase is used to convert glucose to gluconic acid with the production of hydrogen peroxide. The first detection scheme measures oxygen consumption; the second measures the hydrogen peroxide produced by the enzyme reaction; and a third uses a diffusable or immobilized mediator to transfer the electrons from the glucose oxidase to the electrode.

In the case of glucose monitoring, the present device can utilize a sensor which allows glucose and oxygen to diffuse into the enzyme region of the sensor from one direction, but only oxygen diffuses from the other direction. This design helps eliminate the “oxygen deficit”, the low ratio of oxygen to glucose that exists in the body. The modulation of oxygen transport to an oxygen electrode by oxygen participation in the enzyme reaction provides the means for glucose determination. The enzyme catalase is immobilized with the glucose oxidase to remove the hydrogen peroxide, which can shorten the active lifetime of glucose oxidase. This sensing method requires an additional oxygen electrode setup to indicate the background concentration of oxygen.

Hydrogen peroxide sensors measure the product of the enzymatic reaction on an anodically polarized electrode. One of the advantages of hydrogen peroxide sensors is that the signal increases with increasing glucose concentrations. However, the oxidation of hydrogen peroxide requires an applied potential at which many other species commonly found in the body are electro-oxidizable, creating the possibility of interference. The most problematic species are urea, ascorbate (vitamin C), urate, and acetaminophen. Interferences are minimized with semipermeable membranes that restrict their passage. The enzyme reaction still requires oxygen, which is usually assumed to be adequate.

Glucose sensors that use nonleachable electrochemical mediators circumvent the oxygen deficit described above by using a species other than oxygen to transfer the electrons from the glucose oxidase to the electrode. Because oxygen remains in the system, the mediator must compete effectively with the oxygen for the electrons. In the past, ferrocene has been used as a mediator but it is diffusable and toxic. A more recent version of the mediator sensors is the “wired” glucose oxidase electrode designed by Adam Heller and his group in the Department of Chemical Engineering at the University of Texas at Austin. The mediator does not leach because it is bound to a polymer, which is cross-linked. The glucose oxidase is tethered to the electrode with a hydrogel formed of a redox polymer with electrochemically active and chemically bound complexed osmium redox centers.

To ensure long term operation of an electrochemical enzymatic sensor, the present device can be configured capable of “recharging” the sensor with fresh enzyme solution. Such a solution can be pumped into a thin channel between a membrane contacting the bone tissue and the electrode surface. The spent enzyme suspension can be flushed from the system, and fresh enzyme can be injected through a skin port which is in fluid communication with the device

Electrochemical interferences which can affect the accuracy of the analyte readings can be minimized in two ways. The applied potential can be set low enough that few species other than the detected reaction product are oxidized, or a layer that restricts the diffusion of interferences to the electrode can be utilized. In the oxygen-based enzyme sensors, electrochemical interference is much less of a problem because of a pore-free hydrophobic layer between the enzyme and electrode surface that permits oxygen transport but stops polar molecules.

In the case of glucose monitoring, a high-performance glucose sensor, pyrrolo-quinoline quinone dependent glucose dehydrogenase (PQQ-GDH) can be used in the sensor element(s) (U.S. Pat. No. 7,005,048) in order to increase sensor accuracy.

Optical sensors which can be used by the present device include a fluorescent chemical complex immobilized in a thin-film (e.g. thin film hydrogel). The film is a biocompatible polymer which is permeable to the analyte. The sensing system has two components: a fluorescent dye and a “quencher” that is responsive to the analyte. In the absence of the analyte, the quencher binds to the dye and prevents fluorescence, while the interaction of the analyte with the quencher leads to dissociation of the complex and an increase in fluorescence. In such sensors, fluorescence is typically translated into current which is relayed to the monitoring unit

Optical monitoring of glucose can utilize artificial glucose receptors molecules that are fluorescent, such as the compound produced by the coupling of the fluorescent dye, anthracene, to boronic acid, which covalently but reversibly binds to two of the hydroxyl groups on glucose (James T D, Sananayake KRAS, Shinkai S. A glucose-selective molecular fluorescence sensor. Angewandte Chemie International Edition in English. 1994; 33:2207-2209) With this receptor, a change in fluorescence intensity occurs on glucose binding. It also can utilize a NIR light source (Diode/laser etc.) and suitable detectors that measures color changes associated with Glucose fluctuation rates.

Another example of a useful fluorescence technique is “fluorescence resonance energy transfer” (FRET), which relies on the transfer of excitation energy from one fluorescent molecule (the donor) to another nearby molecule (the acceptor) that has overlapping spectral properties. Changes in fluorescence intensity or lifetime are reporters of the changing distance between the donor and acceptor. Model FRET schemes have been described for glucose sensing in vitro with the glucose binding lectin concanavalin A coupled to near infrared fluorescent molecules (olosa L, Szmacinski H, Rao G, Lakowicz J R. Lifetime-based sensing of glucose using energy transfer with a long-lifetime donor. Anal Biochem. 1997; 250:102-108; and Rolinski O J, Birch D J S, McCartney L J, Pickup J C. Near-infrared assay for glucose determination. Soc Photo-optical Instrumentation Engineers Proc. 1999; 3602:6-14).

Conformation change in a protein upon binding of an analyte can also be sensed via a conformation-sensitive fluorophore which is attached to the protein. Molecular engineering techniques are being used in this respect for the rational adaptation of proteins to produce new molecules with modified functions more suited to sensing. For example, conformation sensitive fluorescent groups have been incorporated into allosteric proteins such as the glucose binding protein from Escherichia coli (Marvin J S, Hellinga H W. Engineering biosensors by introducing fluorescent allosteric signal transducers: construction of a novel glucose sensor. J Am Chem Soc. 1998; 120:7-11). This protein undergoes a large conformational change on glucose binding that can be transduced into a change in fluorescence in the engineered protein. Molecular (e.g. nanotube) sensors which react strongly with a chemical such a glucose to change conformation and thus a fluorescent response can also be utilized by the present invention.

Other sensor element(s) configurations which include other sensing mechanisms, including but not limited to biochemical sensors, cell-based sensors (e.g. US 20020038083), electrocatalytic sensors, optical sensors, piezoelectric sensors, thermoelectric sensors, and acoustic sensors can also be used in the present device.

For example, a chemical sensor which permits selective recognition of an analyte using an expandable biocompatible sensor, such as a polymer, that undergoes a dimensional change in the presence of the analyte (see for example, U.S. Pat. No. 6,480,730) can also be used by the present device.

Artificial receptor molecules can also be utilized for analyte monitoring. One of the most promising techniques for creating artificial receptors is called “molecular imprinting” or “plastic antibodies” (Haupt K, Mosbach K. Plastic antibodies: developments and applications. Trends Biotecnol. 1998; 16:468-475.) Monomers that have chemical groups that interact with a template molecule related to the analyte are polymerized around the template, the template is then removed, leaving a polymer that is specific in shape and binding capacity for the analyte. An example for glucose monitoring uses the interaction at alkaline pH between a metal ion complex and glucose, which releases hydrogen ions on glucose binding (Chen G, Guan Z, Chen C-T, Fu L, Sundaresan V, Arnold F. A glucose sensing polymer. Nature Biotechnol. 1997; 15:354-357.) A porous polymer specific for glucose has been made whereby glucose concentration can be measured by titratable release of protons.

Regardless of the sensor type, sensors readings are typically interpreted using circuits such as L-C circuits which are housed within the device of the present invention. For example, the sensor can be coupled to a frequency tuned L-C circuit, where the sensor translates the changes in the physiological condition to the inductor or capacitor of the tuned L-C circuit. Thus, changes in the sensor whether chemical, optical or physical result in changes in the L-C circuit which can be quantified and used to assess analyte concentration.

The present device may include one sensing region, or multiple sensing regions. Each sensing region can be employed to determine the same or different analyte. Different sensing mechanisms may be employed by different sensor regions on the same device.

Although sensor configuration for detection of glucose is exemplified herein, it will be appreciated that any analyte can be detected by the device of the present invention by fitting the system with a sensor (e.g. electrode) designed capable of detecting such an analyte. For example, hydrogen ions (pH) can be detected using an electrode whose output voltage changes as the hydrogen ion concentration changes; hormones can be detected via antibody-based electrodes such as those described by Cook and Devine (Electroanalysis Volume 10, Issue 16, Pages 1108-1111; February 1999) while nitric oxide can be detected by the electrode describe by Mizutani et al. (Chemistry Letters Vol. 29, No. 7 p. 802 2000).

The present device is configured capable of communicating with a remote unit which can be used for controlling the functions of the implanted device, powering it and obtaining readings therefrom. Thus, the present device forms a part of a system for analyte monitoring that further includes a control unit for controlling the operation of the implantable device.

Communication between the implanted device and the control unit can be through wires extending from the device to the control unit; in such cases, the control unit can be implanted under the skin or worn on the body. Communication can also be effected wirelessly, as is further described below.

Powering of the present device can be effected through an implanted power source (which can be integrated into the device) or through remote powering via a remote control unit; remote powering and control of the implanted device is presently preferred.

Several configurations for remote powering and controlling of the present device can be used by the present invention, for a general review of telemetry please see, U.S. Pat. No. 6,201,980.

Inductive coupling of the device and the control unit can be effected through radiofrequency (RF) signals. The implanted device can utilize a first coil which can inductively couple to a second coil provided on the control unit.

During use of the system, the second coil is positioned adjacent the first coil and a high frequency carrier signal is applied to the second coil. The signal is coupled to the first coil, even though there is no direct connection between the two coils, in much the same manner as an AC signal applied to a primary winding of a transformer is coupled to a secondary winding of the transformer. Once received by the first coil, circuitry within the present device rectifies the signal and converts it to a DC signal which is used as the operating power for the implant device. Moreover, modulation applied to the carrier signal provides a means for sending control signals to the implanted device from the control unit. Further description of RF telemetry systems is provided in U.S. Pat. Nos. 6,667,725 and 5,755,748.

Thus, in the case of an electrochemical sensor element(s) and tuned L-C circuitry, a signal transmitted to the coil in the implanted device is converted into a DC current which powers an LC circuit having a frequency which is modulated by the current produced in the sensor electrodes. Such a current is proportional to the amount of analyte present in the environment of the electrodes. Once powered by the signal the LC circuit transmits back to the control unit a frequency modulated signal. The frequency of this signal is interpreted by the control unit to derive an analyte concentration.

Induction coupling for the purpose of powering and controlling the implanted device of the present invention can also be achieved through magnetic (see, for example, U.S. Pat. No. 6,963,779), acoustic (see, for example, U.S. Pat. Nos. 6,764,446 and 7,024,248) or optical telemetry (see, for example, U.S. Pat. Nos. 6,243,608 and 6,349,234) in the case of optical telemetry, a subcutaneous receiver can be wired to the implanted device and serve as a conduit between the device and the extracorporeal control unit. Such a receiver can be a near-infrared light sensor/emitter which converts received light into electrical energy and vice versa.

In any case, telemetry can be used for both controlling and powering of the implanted device.

The control unit can include a user interface for displaying to the user the information relayed by the sensor element(s) of the implanted device. Such information can include the level of the analyte in the blood, trends over a predetermined time period as well as alarms for indicating high or low levels of the analyte. The control unit can store information relating to the subject including analyte level history, personal profile, medications being taken and the like. The control unit can also include an input device such a keypad for inputting information which can be used to set up the system or calibrate it.

The control unit can be in the form of a dedicated wearable device such as a wrist watch, or be integrated into an existing user device such as an MP3 player, a cell phone or the like. Use of a cell phone or other communications-capable device (e.g. computer, PDA) is particularly advantageous since it enables further transmission of the analyte information to a third party over a communications network such as a cellular communication network or a computer network.

The present system can also include an implanted device configuration which includes ports for delivery of medication or alternatively the control unit of the present system can communicate with implanted drug delivery pump or reservoir. Such communication can be though wires or through the telemetry configurations outlined above.

The above described sensor can be integrated into a closed (feedback) loop system which can be used, for example, in controlling blood glucose levels of diabetics. To achieve a closed feedback loop for blood glucose control, a clinically applicable system requires coordination of three components: an implantable insulin pump, an implantable blood glucose sensor, and a control unit which can be implanted or not.

The goal of a fully automatic glucose control system includes prevention or delay of chronic complications of diabetes, lowered risk of hypoglycemia, and less patient inconvenience and discomfort than with multiple daily glucose self-tests and insulin injection.

Implantable insulin pumps which deliver insulin to subcutaneous tissue or a blood vessel such as a vein are feasible for satisfactory control of diabetes for extended time periods. However, closed loop systems employing such implantable pumps are limited by the glucose sensors utilized which provide glucose level readings that are different from real-time blood glucose levels. In addition, subcutaneously implanted insulin pumps are also limited by complications which arise from obstructions in the insulin infusion catheter.

The present inventors postulate that a system which utilizes a bone implanted glucose sensor, such as that described above, in combination with a reservoir having a bone implanted port/catheter would overcome these limitations of prior art systems. Such a system can be a closed loop system in which a signal from the sensor controls an infusion pump, or it can be an open loop system which includes an extracorporeal control unit which receives signals from the sensor and is used (by the subject/physician) to operate the pump accordingly.

Thus, according to another aspect of the present invention there is provided a system for controlling blood glucose levels of a subject.

The system includes the above described bone implanted sensor unit (which in this case is configured for glucose sensing as described above) and a reservoir which receives control signals from the glucose sensor (closed loop) or communicates therewith through an extracorporeal control unit (open loop) and is configured for providing a blood glucose-level modifying composition such as insulin, glucagons, as well as combinations thereof to bone tissue of the subject.

As is further described herein, both the glucose sensor and reservoir are implanted in communication with a bone (preferably skeletal bone) of the subject as is described herein with respect to the analyte sensor described above. The glucose sensor and reservoir are preferably implanted such that each is in communication with a different bone region or a different bone since sensing and infusion in the same bone/bone region can lead to aberrations in blood glucose levels. For example, the glucose sensor can be implanted on one iliac crest and the reservoir on another.

The implanted reservoir can be any implantable reservoir which is capable of delivering insulin and/or other compositions (e.g. glucagons) through a bone infusion port/catheter. Thus, the reservoir can be implanted subcutaneously with a catheter leading to bone tissue, or it can be implanted against bone tissue and anchored thereto with a port leading directly into the bone tissue as is further illustrated in Example 2 of the Examples section which follows.

In any case, the basic configuration of the reservoir includes one or more chambers (each containing a composition), an infusion port/catheter connected thereto and a controllable valve and optionally a pumping mechanism for controlling flow from the reservoir to the port/catheter.

The infusion port/catheter can be anchored into bone tissue as described above for the analyte sensor. To prevent bone ingrowth or local clotting/tissue reactions, the infusion port/catheter can be coated with an anti-clotting composition or bone growth suppressors as described above.

To deliver the composition from the reservoir and through the infusion port/catheter, the pumping mechanism can utilize peristalsis, a propellant, osmotic pressure (e.g. U.S. Pat. No. 6,632,217), a piezoelectric element (e.g. U.S. Pat. Nos. 3,963,380 and 4,344,743), a combination of osmotic pressure and an oscillating piston/rotating turbine and the like.

The pumping mechanism can be utilized to facilitate controlled chamber collapse for delivering the composition contained therein to the bone tissue.

Chamber collapse can be actuated by a mechanical mechanism, an electrically powered mechanism or by using a two-phase fluid, or propellant, that is contained within the housing of the pump in a fluid-tight space adjacent to the composition chamber. Such a propellant is both a liquid and a vapor at patient physiological temperatures, and theoretically exerts a positive, constant pressure over a volume change of the chamber/reservoir, thus effecting the delivery of a constant flow of the composition. When the reservoir is expanded upon being refilled, the propellant is compressed, where a portion of such vapor reverts to its liquid phase and thereby recharges the vapor pressure power source of the pump. Other pump configurations can include a plunger pump mechanism (e.g. Minimed, Medtronic)

Provision of the composition can be as a bolus or a slow infusion. In any case, control of infusion is preferably effected through the valve which is positioned between the reservoir and port/catheter. One configuration of a valve mechanism which can be used by the system of the present invention in variable rate delivery of the composition is described in U.S. 20050054988. Infusion rate is preprogrammed according to the signal received from the sensor and parameters associated with the subject as determined via an examination prior to implantation of the system.

The reservoir can be configured for storing a liquid or a dry preparation of the composition (e.g. insulin).

Since insulin and glucagons have a short half-life as liquid preparations, a reservoir which is configured for storage of a dry (e.g. lyophilized) preparation is presently preferred. A reservoir having such a configuration can include a mechanism for suspending the stored composition in a liquid prior to provision. Such liquefying can be effected by the addition of saline (from a second chamber) or by collection of interstitial fluid (ISF) from the environment surrounding the pump. Alternatively, the reservoir can be configured for direct delivery of a dry composition into the bone in the form of microparticles, such as PLA/PGA microparticles.

Since the system of the present invention is utilized for long term provision of blood glucose level modifying agents, a reservoir utilized thereby might require periodic replenishing. Thus, the reservoir can also include a filling port which can be implanted within the skin. The reservoir may be refilled as needed by an external needle injection through a self-sealing septum provided in a skin port.

As is mentioned hereinabove, the present system can be configured as either a closed loop system or as an open loop system (or a combination of both). In the closed loop configuration, the implanted glucose sensor monitors blood glucose levels and periodically relays glucose readings (e.g. every hour) to the implanted insulin reservoir. The sensor or reservoir can include a processing unit for converting blood glucose level signals to a pump activation signal. Such a processing unit can be accessible from outside the body through a communications port or a wireless communication mode similar to that described above for the implantable analyte sensor and control unit. The processing unit is first calibrated by a physician based on glucose readings and insulin effect as measured by standard tests. The processing unit can be calibrated prior to or following implantation and be recalibrated periodically (e.g. once or several times a year) if need be.

In any case, the signal provided by the glucose sensor is processed and an appropriate infusion-activation signal (amount of insulin, flow rate etc.) is provided.

Implantation and operation of closed loop configurations of the present system is illustrated in Example 2 of the Examples section which follows.

The open loop configuration requires operator control over provision of the composition from the reservoir. As such, the open loop configuration further includes a user operated extracorporeal control unit which is similar in function to the control unit of the analyte sensor described hereinabove. Such a control unit can be used to monitor blood glucose levels and modify infusion rates/composition type periodically.

Control and powering of the pumping mechanism can be as described above for the sensor. A single control and powering unit can be co-implanted with the sensor and reservoir assemblies and provide power and communication for both, as well as processing of sensor and activation signals.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description, illustrate the invention in a non-limiting fashion.

### Example: 1

### Implantation of a Bone-Implanted Electrochemical Glucose Sensor

FIG. 2a illustrates a device 10 which is constructed in accordance with the teachings of the present invention and positioned with bone tissue of a subject. Device 10 includes a housing 20 which houses a sensor element(s) 12 which is connected via circuitry 14 to a power source and telemetry unit 16. Housing 20 can be fabricated from any biocompatible material including polymers, ceramics, alloys and the like. Sensor element(s) 12 is a membrane encapsulated glucose enzyme electrode. Device 10 is positioned such that sensor element(s) 12 extends into bone marrow 24 and as such is exposed to blood flowing therein.

Device 10 is positioned in the bone (e.g. iliac crest) by making an incision in the skin, striping the muscle off the bone. A drill bit is then utilized to drill a hole 26 through the periosteum, cortical bone and cancellous bone layers. Hole 26 is slightly larger in diameter than housing 20 at sensor element(s) 12. Sensor element(s) 12 portion of device 10 is then inserted into hole 26 and positioned such that sensor element(s) 12 is exposed to bone marrow tissue. Housing 20 is then secured against cortical bone 22 via bone screws 18 and the unit is powered tested and calibrated against blood glucose analysis performed using standard laboratory tests. Following calibration, muscle and skin tissue are replaced into position covering device 10 and are sutured or stapled.

### Example 2

### System for Controlling Blood Glucose Levels

FIGS. 3a-b illustrate two configurations of a system for controlling glucose levels constructed in accordance with the teachings of the present invention.

FIG. 3a illustrates a system 50 which includes drug delivery device 52 mounted against the skin of the subject with cannula 54 extending through skin 56 and bone tissue 58 and into bone marrow 60. Cannula 54 conducts fluid from reservoirs 62 and 64 into bone marrow 60 under the driving force of pump 66.

System 50 also includes detector 68 which includes glucose monitor 70 and cannula 72 for conducting blood from bone marrow 60 and into glucose monitor 70 for glucose level assessment. Sensor assembly further includes a reservoir 74 for delivering heparin into bone marrow 60 through cannula 72 under the driving force of pump 76.

Drug delivery device 52 and detector 68 can communicate through a hard wire connection (which can be implanted under the skin of the subject) or through wireless communication through transceivers 80. System 50 is powered in this configuration by a battery 82 (e.g. a Li-ion battery) although other forms of powering including capacitors and coils are also envisaged.

System 50 is positioned as follows: an incision is made above the bone with access obtained to cortical bone. Based on the size of the portion of the device to be inserted into the bone marrow a space is cut through the cortex and into the bone marrow with standard drills and osteotomy tools. The device is then secured with the sensor elements implanted within the bone marrow and the external housing attached to cortical bone by screws.

Following positioning, glucose sensor assembly of system 50 is first calibrated against a standard blood glucose test, following which, reservoirs 62, 64 and 74 are filled via syringes 84 and the system activated. Flow rate of insulin from reservoir 62 of drug delivery device 52 can be determined/adjusted by the subject according to the blood glucose levels determined by glucose monitor 70 and displayed on display 86 or such levels can be automatically determined/adjusted by running system 50 in a closed loop mode, in which case, system 50 will self-adjust insulin flow rates according to glucose monitor 76 readings. Typical insulin delivery rates are in the range of 0.1 unit/hr in young children, to 2-6 units/hr in adults. System 50 also preferably employs shutoff and warning mechanisms to prevent flow rates exceeding optimal levels depending on the body weight, age and typical insulin usage range of the subject.

Drug delivery device 52 can periodically deliver a hormone such as glucagons (10-20 microgram/kg/24 hr) or somatostatin analogues (3-4 mg/kg/day) from reservoir 64 if blood glucose levels drop rapidly towards hypoglycemic levels, as detected by glucose monitor 70. In addition, in order to prevent clogging of cannula 72, a blood thinner/clot dissolver such as heparin can be periodically delivered from reservoir 74 through cannula 72.

In order to maintain glucose control accuracy, system 50 would preferably be calibrated periodically against blood glucose tests.

FIG. 3b illustrates a second configuration of system 50 in which drug delivery device 52 and detector 68 are implanted under skin 56 and anchored against or within bone tissue 58. In this configuration system 50 includes an extracorporeal unit 100 which includes a charger 102 which provides the power to pump and sensors (or to a rechargeable battery connected thereto) and a display 86 for displaying information (e.g. glucose levels) to the subject.

Unit 100 can further provide communication functions to drug delivery device 52 and detector 68 (e.g. coordinating communications therebetween), as well as provide processing of sensor information and relaying of commands to drug delivery device 52. Unit 100 can further include an interface (e.g. keypad) for enabling input of information (e.g. subject information such as weight, operational commands etc.).

An alternative embodiment of system 50 can include the implantable configuration described in FIG. 3b and a pager-like device. Both the detector and the drug delivery device are positioned under the skin and attached to the bone marrow as described above. Each includes a separate internal rechargeable battery thus extending operational time of the system. The pager is placed outside the body and provides data processing and controls insulin glucagon infusion rates etc. Operation of this configuration of system 50 is similar to that described in FIG. 3a.

### Example 3

### Monitoring Glucose Levels in Blood Drawn from a Vein or Bone Marrow of Rabbits

Although tight glycemic control in patients with diabetes has been founded to reduce the risk of micro vascular and macro vascular complications, it is also associated with an increased risk of episodes of severe hypoglycemia. Thus, the ultimate goal in diabetes treatment is to develop an autonomous system (artificial pancreas) capable of continuous glucose sensing and maintaining normal blood glucose levels, thereby mimicking the physiologic function of the islet beta cells and freeing the patient from the need for constant calculations of daily insulin and carbohydrates.

A study was performed in order to compare bone-marrow glucose to blood glucose in healthy and diabetic animals at base line and following insulin or dextrose treatment.

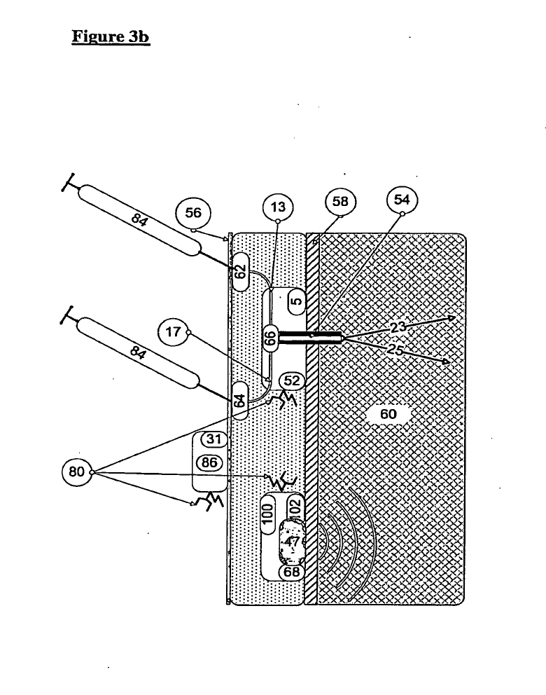

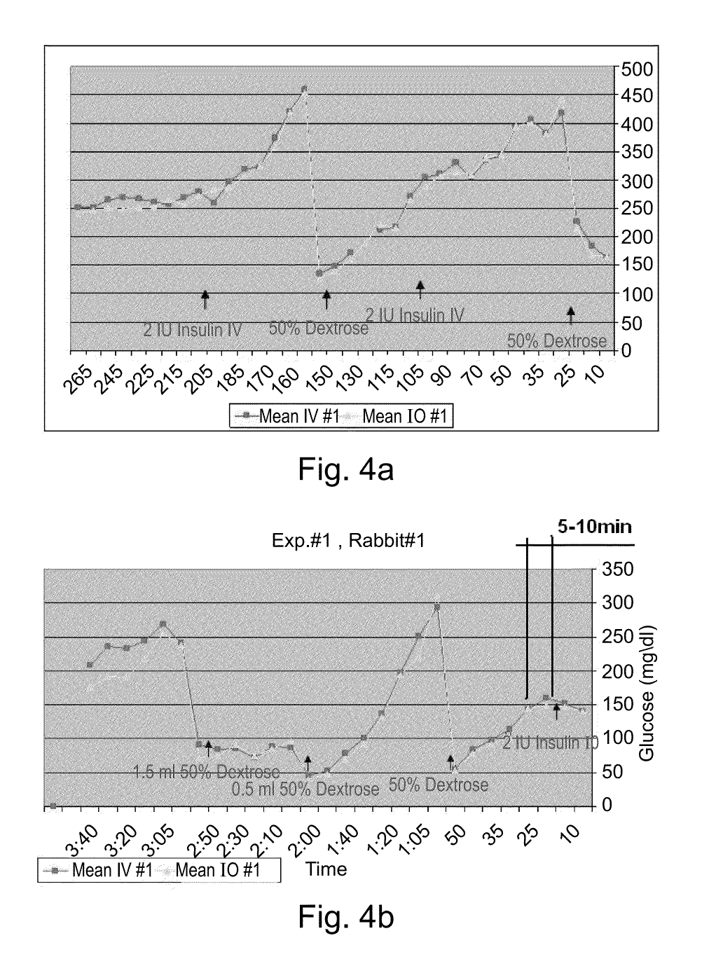

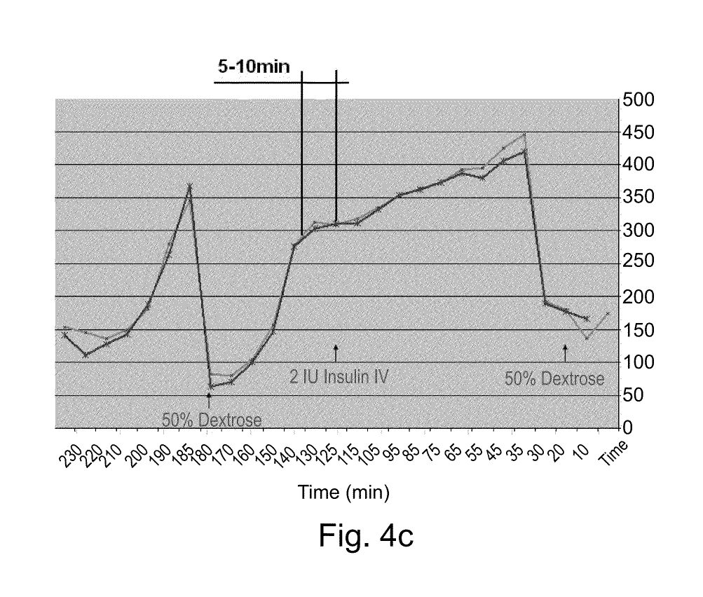

The blood glucose levels of eight adult female rabbits (2 kg each) were manipulated via i.v. infusion of 50% dextrose and 2 IU insulin, the Glucose levels of these rabbits were then measured in vein (IV) and bone (IO) blood (FIG. 4a).

All eight rabbits were subjected to the following phases:
(i) First phase—measurement of steady state glucose level for about 10-30 minutes (sampling every 5-10 min)
(ii) Second phase—Infusion of 50% dextrose
(iii) Third phase—Infusion of 2 IU of insulin (over 3-5 hours)

Samples were obtained from both vein and bone marrow access at the same time in order to correlate glucose levels in blood obtained from both sites

As is clearly shown in FIG. 4a, glucose levels measured in blood drawn from bone marrow track well with glucose levels present in vein blood with a very high correlation level (+−4% error).

The glucose levels in vein and bone marrow derived blood were compared in two rabbits tested with bone marrow insulin infusion (FIG. 4b) and vein insulin infusion (FIG. 4c). Glucose level response to bone marrow delivery of insulin was comparable to that of vein insulin delivery (both reduced glucose levels within 5-10 minutes).

These results clearly illustrate that a system that includes glucose sensing in blood derived from bone as well as insulin delivery into bone blood can be effective in maintaining normal glucose levels and thus can be used in a closed or open loop configuration to treat diabetics.

### Example 4

### Examples of Specific Embodiments of the Cage-Like Device of the Present Invention

The inventors have developed several different versions of the claimed cage-like device, each comprising some or all of the structural features defined and described hereinabove. Several of these different embodiments are illustrated in FIGS. 5-10. In each of these figures, the left-hand pane provides an external view of the illustrated device, while the right-hand pane shows a mid-line longitudinal section thereof. It is to be noted that these particular versions of the claimed device are shown for the purpose of illustration only, and to not limit the scope of the invention in any way.

The device shown in FIG. 5 possesses a single central bore without the addition of any lateral channels. It may be seen from the right-hand panel of this figure (and, indeed, also in FIGS. 6-10) that the upper portion of the central bore has an enlarged internal diameter, immediately distal to which is a region fitted with an internal thread. These two features permit the proximal (upper) central bore of the device to be sealed by means of a small plug or cap (manufactured from either titanium or any other suitable material) screwed into the upper region thereof. Said plug may be used during the implantation of an empty device, and later removed prior to the insertion of an electrode or other implantable element within the central bore. It has been found that the use of this type of plug assists in preventing the undesired ingrowth of fibrotic tissue into the central bore of the device via its proximal (upper) opening.

The device shown in FIG. 6 is fitted with a single through-and-through lateral channel, which (as clearly shown in the longitudinal section) passes from one external surface of the device, through the central bore, finally piercing the external surface on the opposite side. While the specific embodiment shown in FIG. 6 possesses only one through-and-through channel, in other embodiments (not shown), the device may be constructed with two or more such channels.

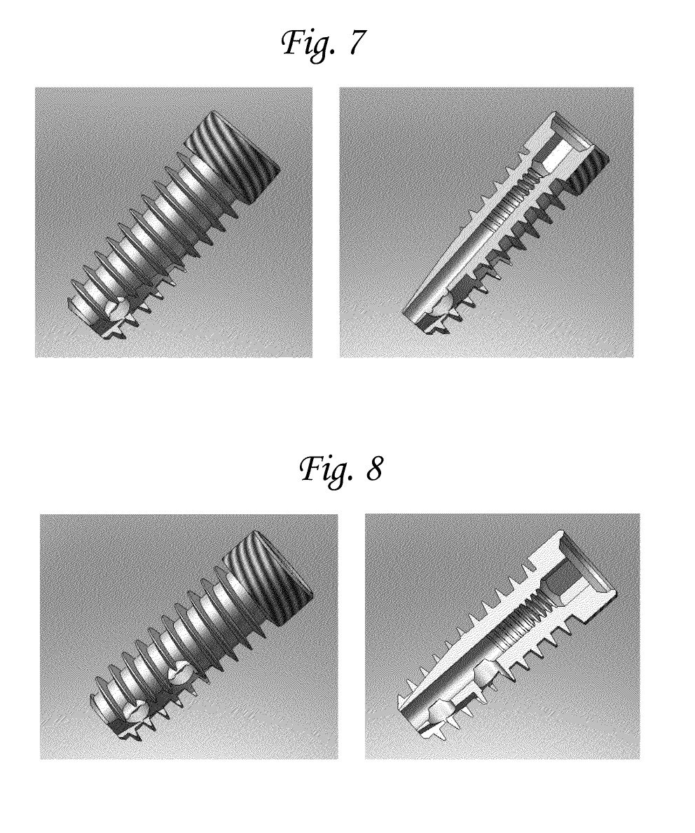

The device shown in FIG. 7 differs from that depicted in the previous figure with regard to the extent of penetration of the lateral channel. Thus, while in the case of the device of FIG. 6, the lateral channel is of the through-and-through type, the channel in the device of FIG. 7 is only partial depth, passing from one external surface of the device internally and ending in the central bore.

The device shown in FIG. 8 contains two partial-depth lateral channels (of the same type as shown in the device of FIG. 7), one above the other. In the specific embodiment shown in this figure, the two channels run parallel to each other. In other embodiments, however, the external openings of the two (or more) channels may be offset, such that said channels run in non-parallel directions.

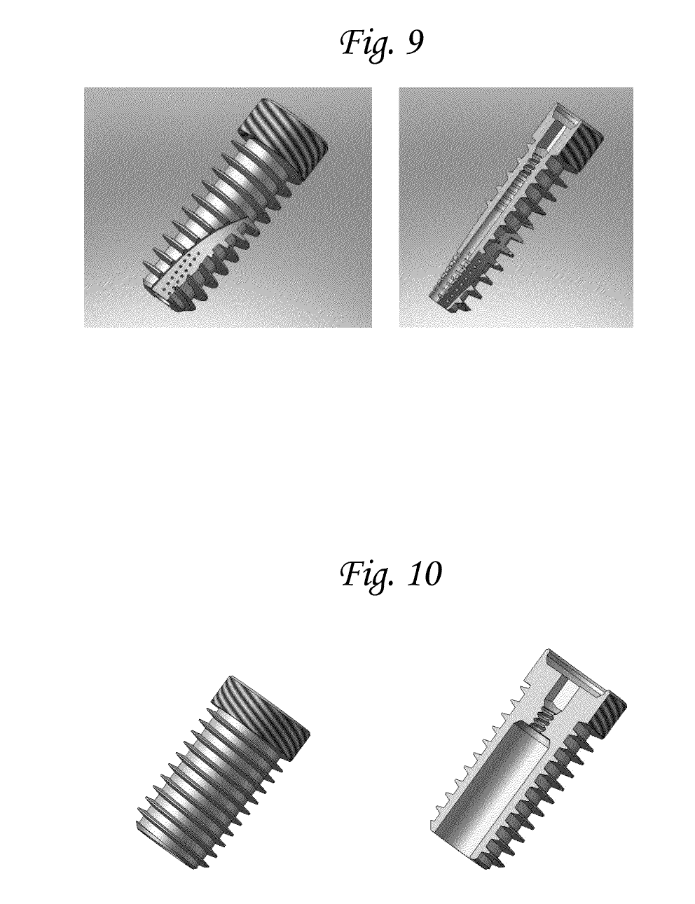

FIG. 9 illustrates an embodiment of the device of the present invention in which the lateral wall has been perforated by an array of 150 micron diameter apertures which were formed by laser drilling. In some embodiments of this type, the micro-aperture array is formed in only a restricted segment of the device wall, thus forming partial-depth channels. In other embodiments however, the array covers the entire circumference of the device wall at a particular height, thereby forming a plurality of lateral channels of the through-and-through type. In a further variation of this embodiment type (not shown), the perforated wall is formed by incorporating a rolled-up perforated sheet into the device (rather than perforating the wall of a pre-existing screw-like device, as shown in FIG. 9).

FIG. 10 depicts a particularly preferred embodiment of the device of the present invention, in which said embodiment is characterized by having the following features:
The entire device is manufactured from titanium.
The external, threaded surface of the device is roughened (either by sandblasting or by incorporation of metallic particles by means of laser welding).
A relatively large-diameter central bore (typically 5 mm).
The internal wall surrounding the central bore may be either polished or coated with an inert coating (such as Excor, produced by Carmeda of Canada), or subjected to both treatments.
The external wall of the device may optionally be perforated by one or more lateral channels (not shown in FIG. 10), each having an internal diameter of 1-2 mm. The internal surface of each of said channels is highly smooth and free of micro-irregularities. In the event that the device is fitted with two more lateral channels, the maximum separation distance between adjacent channels (measured along the external wall of the device) is in the order of 2 mm.
Typical dimensions of this preferred embodiment are:
   Overall height: 16 mm.
   External diameter at proximal (upper) end: 6 mm.
   Length of central bore: 10.5 mm.
   Diameter of central bore: 5 mm.

It is, of course, to be recognized that the dimensions listed above are given only for the purposes of illustrating a best mode embodiment, and do not restrict the scope of the invention in any way.

Further preferred embodiments of the cage device of the present invention are illustrated in FIGS. 16-19, 21 and 23.

FIG. 16 depicts an alternative embodiment, generally indicated as 120 in which the device comprises four additional longitudinal channels 124 contained within the material of the cage wall. These additional channels may be used to house sensor devices such as elongate electrodes, the blood or other tissue fluid to be sampled being able to enter channels 124 via arrays of small-diameter lateral apertures 122 that connect said longitudinal channels with the exterior of the device. In the example of this embodiment depicted in FIG. 16, lateral apertures 122 each have a diameter of 0.2 mm.

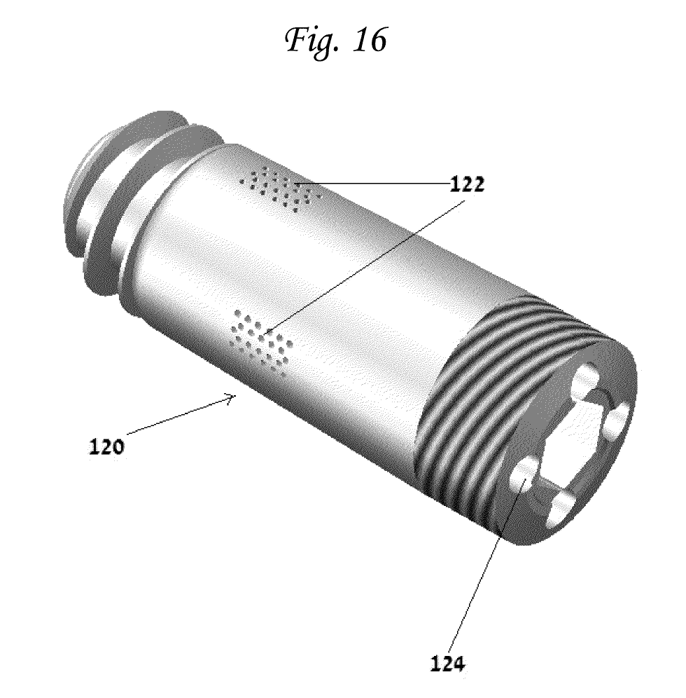

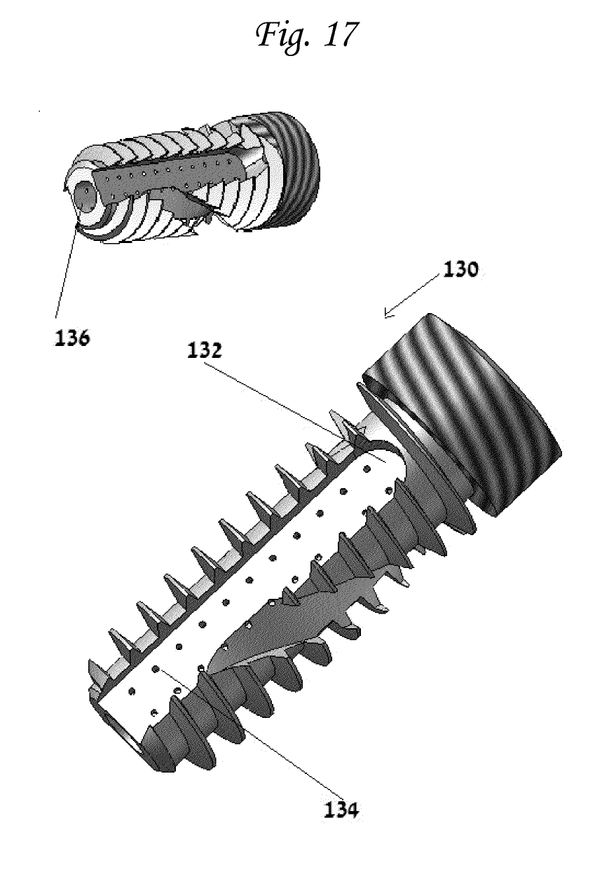

FIG. 17 illustrates another embodiment of the device of the present invention 130, comprising two arrays of 0.2 mm diameter lateral channels 134, in which each array is situated within an exposed slot formed on opposite sides of said device. The longitudinal channel 136 has an internal diameter of 1 mm. As explained hereinabove, and exemplified hereinbelow, this type of device, when implanted within bony tissue, is capable of allowing blood and serum to enter its longitudinal channel by virtue of the restriction of the diameter of each of the lateral channels in the arrays to 0.2 mm. Channels of this diameter are largely unable to permit the ingress of bone marrow tissue into the interior of the cage device.

The device depicted in FIG. 18, represented generally as 140, differs both structurally and functionally from the device shown in FIG. 17. Firstly, device 140 possesses two, large diameter (greater than 0.5 mm) through-and-through lateral channels, 142a and 142b. Such large channels permit, in use, the ingress of bone marrow tissue together with a certain amount of liquid blood. A further feature of this embodiment is the difference in size between the two lateral channels, the distal channel 142a having a larger diameter than proximal channel 142b. It has been found by the present inventors that this type of arrangement is beneficial in that it permits the establishment of a circulatory blood flow, blood and serum entering the device through the large channel 142a and leaving it via the smaller channel 142b. The dynamic flow through the interior spaces of the device that is achieved in this way may prevent undesirable changes in some of the contents of the analyzed blood that may otherwise occur if the blood is allowed to pool in a static manner.

A further example of gradient flow into/out of the device is described in Example 6, hereinbelow.

### Example 5

### Experimental Study I: Implantation of a Device of the Present Invention into Experimental Animals

The aim of this study was to histologically evaluate the host response to implantation of the cage-like devices of the present invention.

Methods:

Commercial pigs having a body weight in the range of 90-120 Kg were used for this study. Prior to surgery, the pigs were fasted overnight and pre-medicated using a Ketamine/Xylasine combination. The animals were anesthetized throughout the procedure. Anesthesia was induced with a Ketamine/Valium mixture and maintained by Isofluorane inhalation. Pulse, oxygen saturation and body temperature were monitored continuously throughout the surgical procedure. The animals were placed in dorsal recumbency and the sternal region was surgically prepared. The first and second sternal bones were then located ultrasonically and marked. A 5 cm ventral midline incision was performed over the sternum, cutting through skin and subcutaneous layers. The sternal bone was then exposed by blunt dissection. Implants were inserted, 1-2 cm apart, into pre-drilled holes and (where necessary, according to the device design used) secured by two screws, one on each side of the device. The implants were removed 8 weeks following their insertion using the following surgical procedure: The animals (anesthetized as described above) were placed in dorsal recumbency and the sternal region surgically prepared. A ventral midline incision through the skin and subcutaneous layers overlying the sternum was then performed, and the first and second sternal bones excised and removed, following which they were fixed, sectioned, stained and subjected to histological evaluation.

Results:

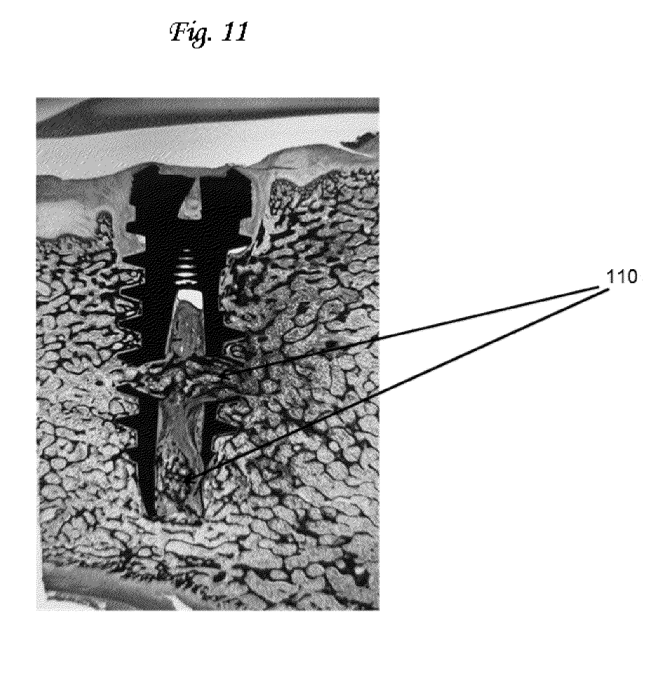

FIGS. 11 to 14 illustrate the histological changes seen in the tissues in contact with devices of the present invention eight weeks following implantation into the sternum. The devices used are of the general type illustrated in FIG. 6 and described hereinabove. In the longitudinal section shown in FIG. 11, it is possible to see the establishment of bone marrow “bridges” 110, which are formed by the ingress of vascular tissue into the central bore of the device via two lateral channels (the position of which are indicated by the black indicator lines in the figure). Very little evidence of the presence of fibrotic tissue within the central bore or lateral channels is seen.

In a further representative longitudinal section shown in FIG. 12, a large portion of the previously empty central bore 112 is now seen to be filled with free-flowing blood.

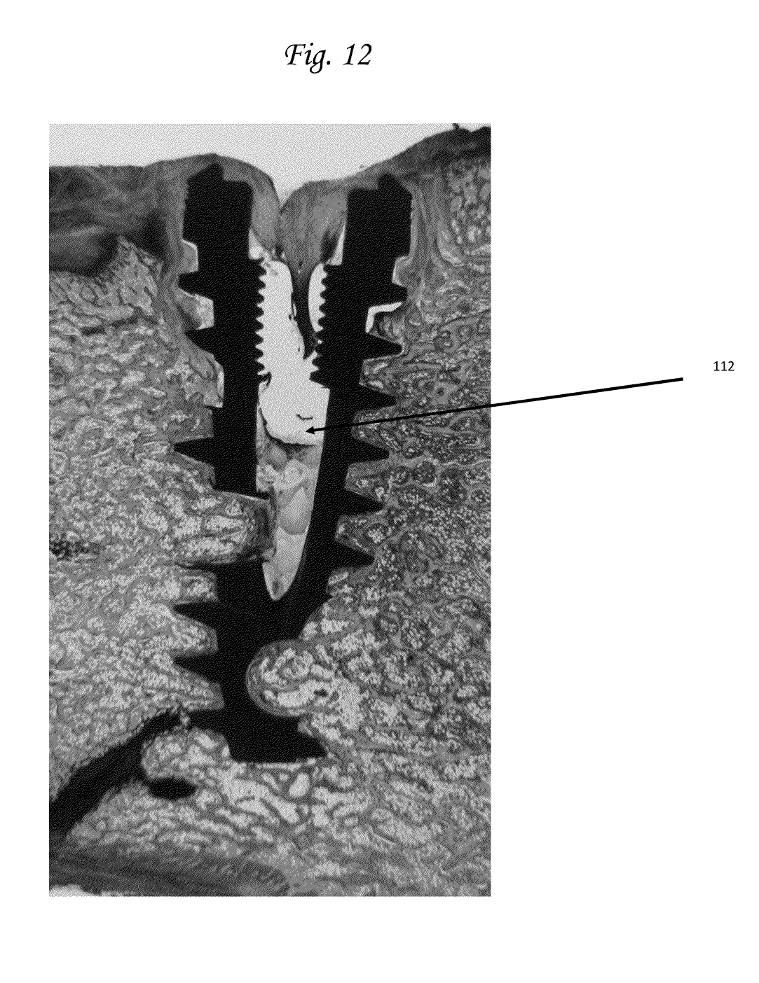

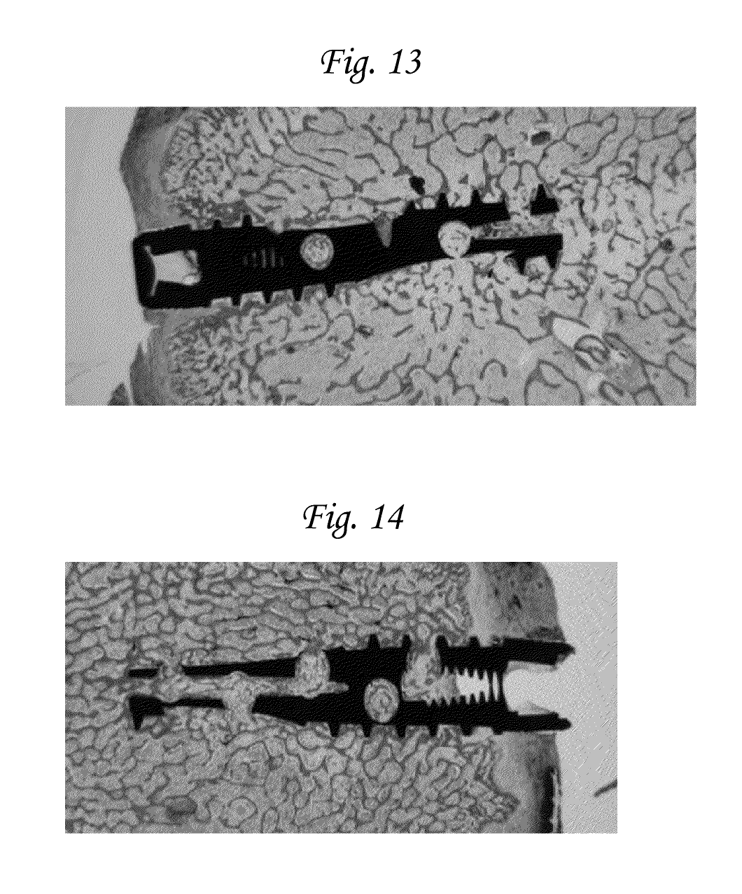

FIGS. 13 and 14 clearly illustrate the fact that, histologically, the bone marrow tissue found within the interior channels and bores of the implanted devices is identical with the bone marrow tissue surrounding said devices—both in terms of its reaction to the histological stain and in terms of the density of said tissue. Furthermore, there are no signs whatsoever of the presence of fibrotic or scar tissue within the interior channels of the devices. Both of these observations indicate that the implantation of the devices was entirely successful.

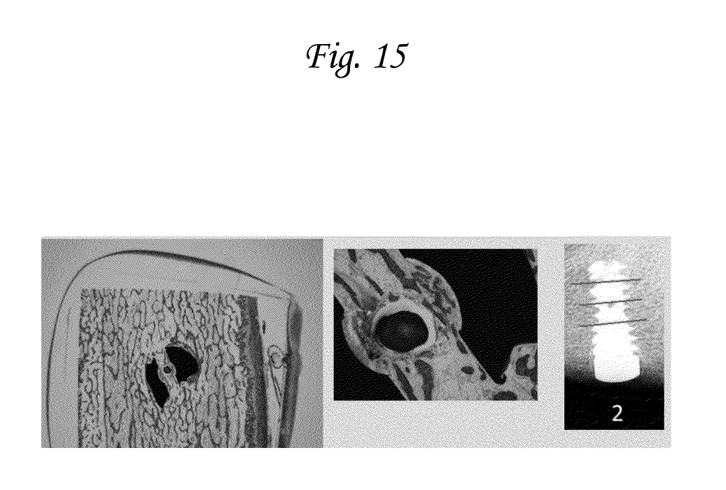

Finally, FIG. 15 presents transverse (horizontal) section views of an implanted cage device. Thus, the left-hand panel illustrates a transverse section taken at a level corresponding to the middle of the three lines indicated in the right-hand panel. This view clearly shows a dark staining central dot (an implanted sensor) surrounded on two sides by the walls of the cage device. Similarly, the central panel shows an enlarged view of this transverse section. It may be clearly seen from both the central and left-hand panels that the tissue within the interior of the implanted cage-like device appears to be histologically identical to the bone marrow tissue surrounding said device.

Conclusions:

The cage-like devices of the present invention encourage the ingrowth of vascular tissue and free-flowing blood within their central bore, thereby ensuring close contact between a sensor or other device placed therein and said vascular tissue and blood. Furthermore, the central bore is largely free from penetration by fibrotic or granulation tissue. These features clearly indicate the suitability of the devices of the present invention for use as protective cages for sensor devices, such as analytical electrodes, placed within their central cavity.

### Example 6

Experimental Study II: Implantation of Devices of the Present Invention into Experimental Animals

The aim of this study was to histologically evaluate the ability of different versions of the device of the present invention to permit blood and/or bone marrow tissue to enter the interior of said device.

The experimental methods used are the same as described in Example 5, hereinabove.

Results:

(A) Small-Diameter Lateral Apertures

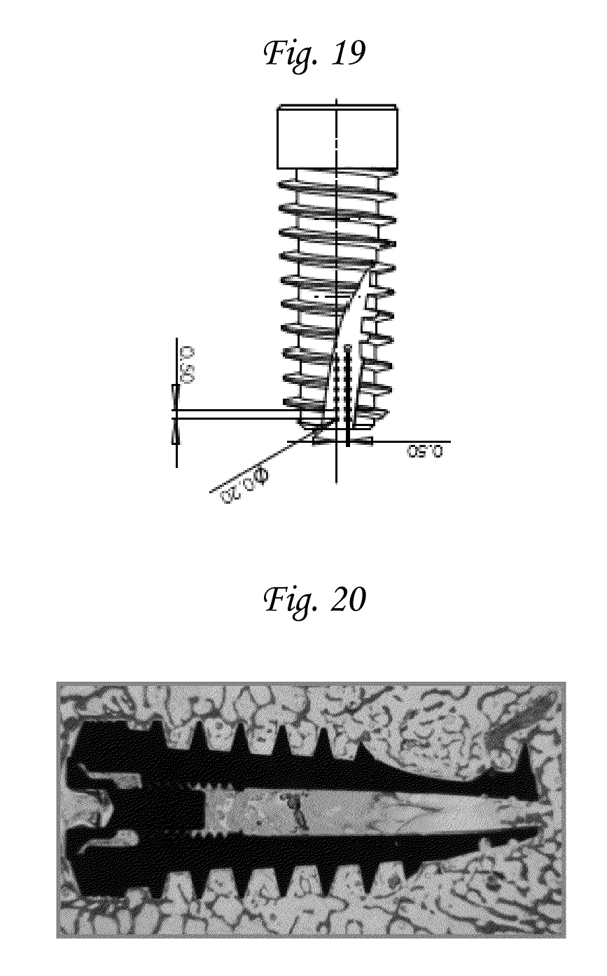

FIG. 19 illustrates an embodiment of the cage device of the present invention in which a laser-cut matrix of 0.2 mm lateral channels is present on the lower part of the side wall of the cage. It may be seen from the stained histological section presented in FIG. 20 that, eight weeks following implantation, only about 15%-20% of the height of the longitudinal channel of the device is occupied by bone marrow and bone trabecula, while the remainder (>80% of the available volume) is filled with liquid (whole blood and serum).

Conclusion:

The small-size lateral apertures (array of 0.2 mm holes) largely prevents the ingress of bone marrow tissue, while freely allowing entry of blood and serum.

(B) Smooth-Bored Large-Diameter Lateral Apertures

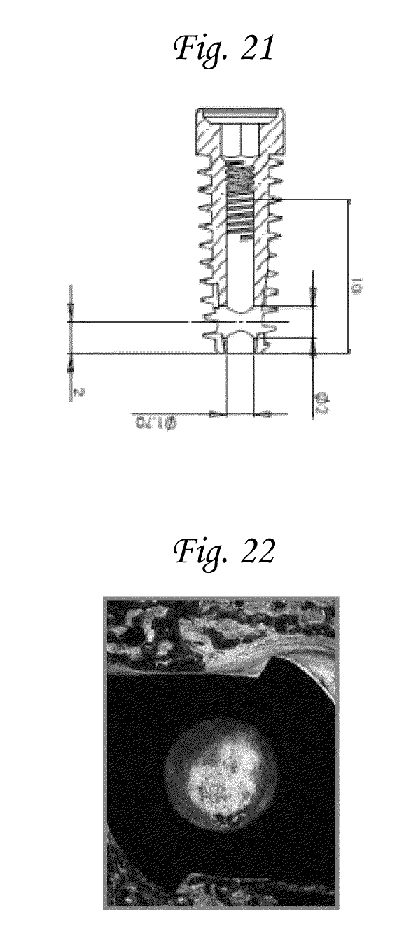

FIG. 21 illustrates a further embodiment of the present invention in which the cage-like device contains a single through-and-through lateral channel, having a diameter of 2 mm, located in the lower portion of said device. The inner walls surrounding both the longitudinal and lateral channels are highly polished. As may be seen in the eight-week histological section shown in FIG. 22, the longitudinal channel of the device has become entirely filled with liquid components (whole blood and serum).

Conclusion:

Despite the relatively large diameter lateral aperture (2 mm) there was no ingress and/or local organization of bone marrow tissue within the internal cavity of the cage, said cavity being entirely filled with blood and serum. This is believed to be due to the highly polished nature of the walls of the longitudinal and lateral channels, which interferes with the ability of bone marrow tissue to physically adhere and establish itself and become organized within the interior of the device.

(C) Laser-Drilled Array Drilled on the Lower End of Cage and Four Larger Apertures in the Middle Portion Thereof.

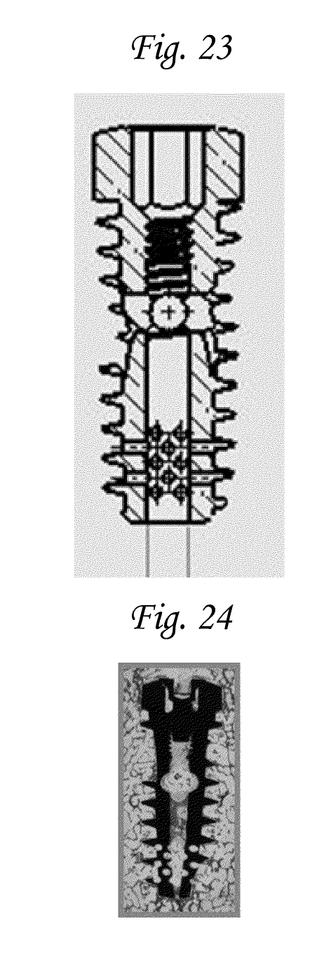

FIG. 23 illustrates an embodiment of the present invention in which four large-diameter lateral apertures are present in the upper portion of the device, while the lower portion of the device contains an array of small, laser-drilled apertures.

FIG. 24 is a histological section taken from tissue obtained eight-weeks following implantation of the device shown in FIG. 23. It may be seen from this section that 80% of the interior space of the device is filled with blood and bone marrow, while about 20% consists of bone trabecula and fibrosis. Generally, there is a homogeneous distribution of bone marrow and blood throughout the entire volume of the lumen.

Conclusion:

The presence of large diameter lateral apertures at one end of the cage and small diameter (e.g. laser-cut array of 0.2 mm holes) at the other, leads to a favorable situation in which a pressure—and hence—flow gradient is established. Thus, in the case of this implant, blood flows into the cage implant through the upper, large diameter apertures, descends through the central cavity, and then exits the device via the lower micro-apertures. The circulatory flow that is thereby established is highly advantageous from the point of view of providing fresh blood for assay, and is clearly greatly superior to a static, potentially stagnant pool of blood that may otherwise collect in the internal cavity of the device. Furthermore, the presence of the large-diameter apertures permits the ingress of bone marrow tissue into the interior of the device, thereby providing a means for producing fresh blood components in situ.

In addition, continuous flow and high pressure of fluid, blood inhibits the establishment of tissue growth, formation of blood clots and accumulation of metabolites which influence sensor activity (e.g. low oxygen tension), i.e. essentially this form of implant act as a vessel by which blood, fluid or tissue, flow rate, and pressure could be altered with pores bore size, position, location, placement and the angle by which the edges of each lateral channel is cut in the implant.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A system for monitoring an analyte in a subject comprising a sensor element being designed and configured for detecting said analyte in blood flowing through a bone of the subject, and a fixation element that is capable of fixating said sensor element within the bone tissue.

2. A system for monitoring an analyte in a subject comprising a sensor element being designed and configured for detecting said analyte in blood flowing through a bone of the subject, and a rigid implant-protecting and stabilizing device comprising an elongate body perforated by a longitudinally-disposed central bore, wherein said device is of a size and shape such that it is capable of completely or partially containing said sensor element within its central bore.

3. A device for monitoring an analyte in a subject comprising a sensor element being designed and configured for detecting the analyte in blood flowing through a bone of the subject.

4. The device of claim 3, wherein the device is completely implanted within tissue of the subject.

5. The device of claim 4, wherein said sensor element is implanted within bone tissue and is designed and configured for contacting blood flowing within a blood sinus of said bone tissue.

6. The device of claim 3, wherein said sensor element is anchored to bone tissue.

7. The device of claim 3, further comprising a wireless communication unit for remotely communicating with a wireless control unit.

8. The device of claim 3, further comprising circuitry for remotely powering said sensor element.

9. The device of claim 3, wherein said analyte is glucose.

10. A system for monitoring an analyte in a subject comprising a device including a sensor element being designed and configured for detecting the analyte in blood flowing through a bone of the subject and a reservoir for providing at least one composition capable of modifying a level of the analyte in said blood flowing through said bone of the subject.

11. The system of claim 10, wherein said sensor element is implanted within bone tissue and is designed and configured for contacting blood flowing within a blood sinus of said bone tissue.

12. The system of claim 10, further comprising a wireless control unit for wirelessly controlling said device

13. The system of claim 10, wherein said analyte is glucose.

14. The system of claim 12, wherein said wireless control unit is capable of closed loop operation.

15. The system of claim 10, further comprising a mechanism for pumping said composition from said reservoir to said blood flowing through said bone.

16. The system of claim 10, wherein said reservoir further includes a filling port.

17. The system of claim 10, wherein said at least one composition is insulin or glucagon.

18. A method of controlling a blood glucose level in a subject in need comprising determining a glucose level of the subject in need in blood flowing through bone tissue and if needed, administering an appropriate amount of insulin or glucagon to the subject in need to control the blood glucose level.

19. The method of claim 18, wherein said determining said glucose level is effected via a glucose sensor implanted within bone tissue of the subject.

20. The method of claim 18, wherein said bone is an iliac crest bone.

21. The method of claim 18, wherein said administering an appropriate amount of insulin is effected via an insulin containing reservoir implanted in tissue of the subject in need.

22. The method of claim 18, wherein said administering is effected automatically under closed loop control.

23. A rigid implant-protecting and stabilizing device comprising an elongate body perforated by a longitudinally-disposed central bore, wherein said device is of a size and shape such that it is capable of completely or partially containing a sensor element within said central bore.

24. The device according to claim 23, wherein the central bore is configured such that it is capable of forming a reservoir for a biological fluid within said lumen following implantation into the tissue, said reservoir being defined at least in part by said tissue.

25. The device according to claim 23, further comprising a screw thread on its external surface.

26. The device according to claim 23, further comprising at least one lateral channel that is orientated such that it is not parallel to the longitudinal axis of said device.

27. The device according to claim 26, wherein the at least one lateral channel is orientated at approximately right angles to the longitudinal axis of said device

28. The device according to claim 26, wherein the at least one lateral channel is a partial length channel that pierces the external wall of said device at one side and terminates internally within the central bore.

29. The device according to claim 26, wherein the at least one lateral channel is a through-and-through channel that pierces the external wall of said device on side thereof, passes through the central bore and pierces the external wall of the device on the other side thereof.

30. The device according to claim 26, wherein said device comprises at least two lateral channels, and wherein the distance between adjacent lateral channels is no greater than 5 mm.

31. The device according to claim 30, wherein the distance between adjacent lateral channels is no greater than 2 mm.

32. The device according to claim 26, wherein the internal diameter of the lateral channels is in the range of 0.25-5.0 mm.

33. The device according to claim 26, wherein said device comprises an array of small-diameter lateral channels, each of said channels having an internal diameter in the range of 200 to 500 μm.

34. The device according to claim 26, wherein the internal diameter of the lateral channels is in the range of 0.25-5.0 mm.

35. The device according to claim 23, wherein said device further comprises one or more additional longitudinal channels.

36. The device according to claim 26, wherein the internal walls surrounding the central bore and/or lateral apertures are polished.

37. The device according to claim 26, wherein the internal walls surrounding the central bore and/or lateral apertures are roughened.

38. The device according to claim 26, wherein the internal walls surrounding the central bore and/or lateral apertures are coated with an anti-fibrotic coating.

39. A system for implanting a sensor element within a body tissue, comprising an implant-protecting and stabilizing device according to claim 23 and a sensor element, wherein said sensor element is capable of being inserted, either fully or partially, within an internal channel of said implant-protecting device.

40. A system according to claim 39, wherein the sensor element is a glucose-sensing element.

41. A method for implanting a sensor element in a protected environment within bone tissue comprising the steps of:
a) gaining surgical access to the desired implant site;
b) positioning in the bone a tissue implant including an elongate body having a lumen of a size and shape selected for completely or partially containing a sensor element such that said lumen is in fluid communication with the tissue; and
c) inserting said sensor element into said lumen.

42. The method according to claim 41, wherein said lumen is configured such that it is capable of forming a reservoir for a biological fluid within said lumen following implantation into the tissue, said reservoir being defined at least in part by cells of the tissue.

43. A method for implanting a sensor element in a protected environment within bone tissue comprising the steps of:
a) gaining surgical access to the desired implant site;
b) drilling a hole of appropriate size in said implant site in order to accommodate an implant-protecting and stabilizing device comprising one or more longitudinal channels;
c) inserting said sensor element into a longitudinal channel of said implant-protecting device;
d) inserting the implant-protecting and stabilizing device with the pre-inserted sensor element into said drilled hole;
wherein said protected environment permits blood and bone marrow to come into contact with said sensor element, while preventing, either fully or partially, contact of said sensor device with fibrotic tissue.

44. The method according to either claim 41 or claim 43, wherein the sensor element is a glucose-detecting sensor.

45. An implantable device comprising a tissue anchoring element having at least one internal lumen configured such that it is capable of forming a reservoir for a biological fluid following implantation into the tissue, the boundaries of said reservoir being defined at least in part by the cells of said tissue.

46. The implantable device according to claim 45, wherein the at least one internal lumen is configured for:
(i) limiting migration of cells and tissue into said lumen; and
(ii) enabling the flow of biological fluid into said lumen.

47. The system according to claim 1, wherein the fixation element comprises a porous band that is capable of promoting ingrowth of bony tissue to provide fixation to the bone and fixating the sensor element within the band and bone marrow.

48. The system according to claim 1, wherein the fixation element comprises a screw that is capable of being attached to cortical bone and to the sensor element.

49. The system according to claim 1, wherein the fixation element comprises a fixation plate that is capable of being attached to cortical bone and to the sensor element.
